SEQUENCE LISTING (6 sequences)

SEQ ID NO: 1 - DNA, 21 bp, artificial sequence
  primer
ttccctgtaaaagcctcaatt

SEQ ID NO: 2 - DNA, 25 bp, artificial sequence
  primer
gctcttctccgttgcttttggtcat

SEQ ID NO: 3 - DNA, 19 bp, artificial sequence
  primer
aagaccaaaagcaacggag

SEQ ID NO: 4 - DNA, 19 bp, artificial sequence
  primer
tgctggggttaaaggtgtg

SEQ ID NO: 5 - DNA, 27 bp, artificial sequence
  primer
aggcgcgcccagggcgcagcggcctct

SEQ ID NO: 6 - DNA, 28 bp, artificial sequence
  primer
ttcccccgggatggcgtggggaacgccc

(19) United States
(12) Patent Application Publication
(10) Pub. No.: US 2007/0259825 A1
(43) Pub. Date: Nov. 8, 2007

(54) Transgenic Mammals and Cell Lines for the Identification of Glutamate Transporter Modulators

(75) Inventors: Jeffrey D. Rothstein, Cantonsville, MD (US); Melissa Regan, Towson, MD (US); Shuy Vang Toan, Paris (FR)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Johns Hopkins University, Baltimore, MD (US)

(21) Appl. No.: 11/661,624

(22) PCT Filed: Aug. 31, 2005
(86) PCT No.: PCT/US05/30816

§ 371 Date: Mar. 6, 2007

Related U.S. Application Data
(60) Provisional application No. 60/606,395, filed on Aug. 31, 2004.

(51) Int. Cl. A01K 67/027; A61K 31/70; C12N 5/06; C12Q 1/68

(52) U.S. Cl. 514/44; 435/352; 435/6; 800/14

(57) ABSTRACT

The invention includes a transgenic, non-human mammal useful for identifying candidate compounds for treating neurological and/or psychiatric disorders. Incorporated into the genome of the transgenic mammal is a transgene comprising a glutamate transporter promoter operatively linked to a reporter gene. The transgenic non-human mammal and cells isolated therefrom can be used as an in vivo model for the identification of candidate compounds useful for the treatment of neurological and/or psychiatric disorders.

### STATEMENT REGARDING FEDERALLY SPONSORED RESEARCH OR DEVELOPMENT

This invention was made, in part, using funds obtained from the U.S. Government (National Institutes of Health Grant No. NS33958), and the U.S. Government may therefore have certain rights in this invention.

### FIELD OF THE INVENTION

The present invention features BAC transgenic non-human mammals and cells comprising a glutamate transporter promoter operatively linked to a reporter gene. The present invention further provides methods for identifying compounds useful in treatment of neurological and/or psychiatric disorders.

### BACKGROUND OF THE INVENTION

Glutamate and aspartate are the predominant excitatory neurotransmitters in the mammalian central nervous system (CNS). These excitatory amino acids (EAAs) activate ligand-gated ion channels that are named for the agonists N-methyl-D-aspartate (NMDA), alpha-amino-3-hydroxy-5-methyl-4-isoxazole propionate (AMPA), and kainate and G-protein-coupled metabotropic receptors (Ehlers et al., 1996, Curr. Opin. Cell. Biol. 8:484-489). Paradoxically, there is substantial evidence that an extracellular accumulation of EAAs and excessive activation of EAA receptors also contributes to the neuronal cell death observed in acute insults to the CNS (Choi, 1992, J. Neurobiol. 1261-1276). The process known as “excitotoxicity” may also contribute to neuronal loss observed in chronic neurodegenerative diseases, including amyotrophic lateral sclerosis (ALS). Excitotoxicity is based on altered extracellular concentrations of the EAA, since it is this pool that can be toxic to neurons. The intracellular concentrations of glutamate (about 5-10 mM) and aspartate (about 1-5 mM) are 1000-fold to 10,000-fold greater than the extracellular concentrations (<1-10 μM). Importantly, low extracellular levels of glutamate are maintained by transport into neurons and astrocytes (Danbolt, 2001, Prog. Neurobiol. 65:1-105).

Five distinct glutamate transporters have been cloned that express sodium-dependent high-affinity transporter proteins and include: GLT-1 (known as Excitatory Amino Acid Transporter 2, or EAAT2, in humans) (Arriza et al., 1993, J. Biol. Chem. 268:15329-15332; Pines et al., 1992 Nature 360:464-467), EAAC1/EAAT3 (Arriza et al., 1993, J. Biol. Chem. 268:15329-15332; Kanai et al., 1996, In: Neurotransmitter Transporters: Structure, Function and Regulation, Reith, M. E. A., ed., Humana Press Inc., Totowa N.J., 171-213), GLAST/EAAT1 (Arriza et al., 1993, J. Biol. Chem. 268:15329-15332; Storck et al., 1992, Proc. Natl. Acad. Sci. USA 89:10955-10999), EAAT4 (Fairman et al., 1995, Nature 375:599-603; Lin, C. L. G. et al., 1998, Mol. Brain. Res. 63:174-179), and EAAT5 (Arriza et al., 1997, Proc. Natl. Acad. Sci. USA 94:4155-4160; Danbolt, 2001, Prog. Neurobiol. 65:1-105). There is also evidence for additional heterogeneity of GLT-1 and GLAST mRNA that originates from alternative mRNA splicing. Expression of EAAT1/GLAST and EAAT2/GLT-1 is generally restricted to astroglia. Expression of EAAT3/EAAC 1 and EAAT4 is restricted to neurons, while EAAT5 is restricted to the retina (for review, see Danbolt, 2001, Prog. Neurobiol. 65: 1-105).

Although it was once thought that presynaptic transporters had a major role in the clearance of EAAs during synaptic transmission, it is now known that the astroglial transporters EAAT2/GLT-1, and to a lesser extent, EAAT1/GLAST, are responsible for the bulk of glutamate transport. Support for this includes: 1) calculations suggest that GLT-1 represents approximately 1% of total brain protein (Danbolt et al. 2001, Prog. Neurobiol. 65:1-105); 2) anti-sense knock-down of GLT-1 and/or tissue from GLT-1 null mice suggest that this transport may account for up to 95% of total glutamate transport activity, although its contributions vary by brain region (Rothstein et al., 1996, Neuron 16:675-686; Tanaka et al., 1997, Science 276:1699-1702); and 3) electrophysiological recording of transporter mediated currents in brain preparations strongly suggests that GLT-1 has a primary role for the clearance of glutamate during synaptic transmission in the forebrain (Bergles et al., 1997, Proc. Natl. Acad. Sci. USA 94:14821-14825; Bergles and Jahr, 1997, Neuron 19:1297-1308; Bergles et al., 1998, J. Neurosci. 18:7709-7716; Danbolt, 2001, Prog. Neurobiol. 65:1-105; Diamond et al., 1998, Neuron 21:425-433).

The expression of GLT-1 (EAAT2) is dynamically regulated both in vivo and in vitro. Although GLT-1 is the predominant transporter in the adult CNS, expression is rather low early in development and increases during synaptogenesis in both rats and humans (Furuta et al., 1997, J. Neurosci. 17:8363-8375; Sutherland et al., 1996, J. Neurosci. 16:2191-2207). Lesions of neurons, especially loss of presynaptic terminals leads to selective—but transient—downregulations of GLT-1 and GLAST expression (Ginsberg et al., 1995, J. Neurochem. 65:2800-2803; Ginsberg et al., 1999, Neuroscience 88:1059-1071). These data suggest that the presence of neurons participate in the expression of GLT-1. Several different groups have demonstrated decreased expression of GLT-1 and/or GLAST in mammal models of acute insults to the CNS, including stroke and traumatic brain injury (for review, see Danbolt, 2001, Prog. Neurobiol. 65:1-105). Decreases in EAAT2/GLT-1 expression has been observed repeatedly in patients with ALS (Fray et al., 1998, Eur. J. Neurosci. 10:2481-2489; Rothstein et al., 1992, N. Engl. J. Med. 326:1464-1468; Rothstein et al., 1995, Ann. Neurol. 38:73-84) and in the mutant SOD1 mouse models (Bruijn et al., 1997, Neuron 18:327-33; Keller et al., 1997, Neuroscience 80:685-696; Kruman et al., 1999, Exp. Neurol. 160:28-39), and the mutant SOD1 G93A rat model (Howland et al., 2002, Proc. Natl. Acad. Sci. USA 99:1604-1609). However, not all CNS diseases associated with loss of neurons are associated with loss of GLT-1.

Even though GLT-1 expression is extremely high in vivo, “normal” astrocytes maintained in culture express essentially no detectable protein. Interestingly co-culturing astrocytes with neurons induces glial expression of GLT-1, suggesting that neurons induce and/or maintain expression of GLT-1 in vitro (Schlag et al., 1998, Mol. Pharmacol. 53:355-369; Swanson et al., 1997, J. Neurosci. 17:932-940). This effect of neurons is, at least in part, mediated by a soluble secreted molecule (Schlag et al., 1998, Mol. Pharmacol. 53:355-369). Several small molecules mimic this effect of neurons, including dbcAMP, epidermal growth factor, pituitary adenylate cyclase-activating peptide, and immunophilin (Duan et al., 1999, J. Neurosci. 19:10193-10200; Gegelashvili et al., 1997, J. Neurochem. 69:2612-2615; Rodriguez-Kern et al., 2003, Neurochem. Int. 43:363-370; Schlag et al., 1998, Mol. Pharmacol. 53:355-369; Swanson et al., 1997, J. Neurosci. 17:932-940). In all cases, the increased GLT-1 protein expression was accompanied by increased GLT-1 mRNA and altered astrocyte morphology that is similar to that observed with astrocyte differentiation. However, increased expression does not always lead to increased functional activity; trafficking of the transporter protein to the cell surface is also necessary. These studies suggest that astrocyte cultures provide a valuable model system to determine how neurons regulate GLT-1 expression.

It has shown that the effects of dbcAMP are blocked by an inhibitor of protein kinase A (Schlag et al., 1998, Mol. Pharmacol. 53:355-369; Zelenaia et al., 2000, Mol. Pharmacol. 57:667-678) and that the increase in GLT-1 expression induced by dbcAMP, epidermal growth factor, or neuron conditioned medium are all blocked by an inhibitor of either phosphatidylinositol 3-kinase (PI3-kinase) or an inhibitor of the transcription factor NFκB (Schlag et al., 1998, Mol. Pharmacol. 53:355-369; Zelenaia et al., 2000, Mol. Pharmacol. 57:667-678). Except for this information, very little is known about the mechanisms that actually control GLT-1 expression.

GLAST (EAAT1) on the other hand, exhibits decreased expression throughout development, but in adulthood is still the major glutamate transporter in the cerebellum, where it is expressed by Bergmann glia, and in the cochlea, where it is expressed by glia surrounding inner hair cells.

Studies indicate that GLAST helps to limit the activation of AMPA and metabotropic glutamate receptors in GABAergic interneurons of the hippocampus and cerebellum (Lopez-Bayghen et al., 2003, Brain Res. Mol. Brain Res. 115:1-9). Thus, changes in the abundance of GLAST may lead to changes in the size and duration of responses at many excitatory synapses. Although the expression of GLAST increases during development and is also altered in neurological and/or psychiatric diseases, the mechanisms that regulate GLAST expression in vivo are not well understood.

Changes in protein expression are generally controlled by transcriptional regulation of the encoding gene via interaction of regulatory molecules with the gene's promoter, an ill-defined region of DNA generally, but not necessarily exclusively, upstream of the start codon. Modulation of glutamate transporters presents an avenue for the identification of effective therapies for treating neurological and/or psychiatric disorders. Accordingly, there exists a need in the art for methods and compositions capable of modulating glutamate transporter promoters, as well as for identifying compounds which can modulate glutamate transporter promoters.

### SUMMARY OF THE INVENTION

The present invention is based, at least in part, on the generation of novel non-human transgenic mammals and cells for use in identifying compounds useful for the treatment of neurological and/or psychiatric disorders.

In one embodiment, the invention includes non-human transgenic mammals comprising a bacterial artificial chromosome (BAC) transgene, wherein the BAC comprises genomic DNA containing either the GLT-1 locus or the GLAST locus, as well as a reporter gene that is operatively linked to the promoter of the locus. In a preferred embodiment, the BAC is derived from the same species as the non-human transgenic mammal.

Preferably, the non-human transgenic mammal is a mammal, more preferably a mouse. In another preferred embodiment, the BAC comprises BAC clone RPCI-23-361H22 or BAC clone RPCI-24-287G11.

In another preferred embodiment, the reporter gene comprises a nucleic acid sequence encoding at least one protein selected from the group consisting of luciferase, β-galactosidase, chloramphenicol acetyl transferase and a fluorescent protein (e.g., green fluorescent protein, enhanced green fluorescent protein, red fluorescent protein, yellow fluorescent protein, blue fluorescent protein, or cyan fluorescent protein).

In another embodiment, the invention features cells (e.g., primary or immortalized cells) isolated from the non-human transgenic mammals of the invention. In one embodiment, the cells are astrocytes, preferably the astrocytes exhibit bacterial artificial chromosome GLT-1 promoter activity. In another embodiment, the cells are oligodendrocytes, preferably the oligodendrocytes exhibit bacterial artificial chromosome GLT-1 promoter activity. In yet another embodiment, the cell exhibits bacterial artificial chromosome GLT-1 and bacterial artificial chromosome GLAST promoter activity.

In another embodiment, the invention features methods of identifying a compound capable of treating neurological and/or psychiatric disorders, and/or capable of modulating the GLT-1 and/or GLAST promoter, comprising contacting the cells of the invention with a test compound, and determining whether expression of the reporter gene is modulated, thereby identifying a compound which modulates the GLT-1 and/or GLAST promoter as a compound which is capable of treating a neurological and/or psychiatric disorder. In one embodiment, the test compound increases reporter gene expression. In another embodiment, the test compound decreases reporter gene expression.

In one embodiment, expression of the reporter gene is detected by measuring the level of reporter gene mRNA (e.g., by Northern blotting, RT-PCR or quantitative RT-PCR, primer extension, or nuclease protection).

In another embodiment, expression of the reporter gene is detected by detecting the polypeptide encoded by the reporter gene (e.g., by Western blotting, ELISA, or RIA). In another embodiment, the polypeptide is detected by measuring luciferase activity (e.g., by using a standard luciferase assay). In another embodiment, the polypeptide is detected by measuring chloramphenicol acetyl transferase activity (e.g., using a standard chloramphenicol acetyl transferase assay). In still another embodiment, the polypeptide is detected by measuring the fluorescence of the fluorescent protein.

In another embodiment, the invention provides methods of identifying a compound capable of treating a neurological and/or psychiatric disorder, and/or capable of modulating the GLT-1 and/or GLAST promoter, comprising contacting the non-human transgenic mammal of the invention with a test compound, and determining whether expression of the reporter gene is modulated, thereby identifying a compound which modulates the GLT-1 and/or GLAST promoter as a compound which is capable of treating a neurological and/or psychiatric disorder. In a preferred embodiment, the test compound is administered to the mammal via intrathecal, intravenous, intramuscular, transdermal, or intraperitoneal injection, or via ingestion.

The invention also includes a method of treating a mammal suffering from a neurological and/or psychiatric disease or disorder comprising administering to the mammal a compound capable of increasing the activity of a glutamate transporter promoter. In another embodiment, the method comprises administering to the mammal a compound capable of decreasing the activity of a glutamate transporter promoter.

In another embodiment, the invention provides a method of isolating a cell from a non-human transgenic animal comprising a bacterial artificial chromosome transgene, wherein the BAC comprises genomic DNA comprising a locus selected from the group consisting of the GLT-1 locus and the GLAST locus, as well as a reporter gene that is operatively linked to the promoter of the locus. The method comprises providing an antibody specific for the reporter gene to a population of cells, contacting the population of cells with the antibody under conditions suitable for formation of an antibody-cell complex, and separating the antibody-cell complex from the population of cells, thereby isolating the cell of interest.

In one embodiment, the antibody is conjugated to a physical support. Preferably, the physical support is selected from the group consisting of a microbead, a magnetic bead, a panning surface, a dense particle for density centrifugation, an adsorption column and an adsorption membrane. In yet another embodiment, the physical support is selected from the group consisting of a streptavidin bead and a biotin bead.

In another embodiment, the antibody-cell complex is separated from said population of cells using a method selected from the group consisting of fluorescence activated cell sorting (FACS) and magnetic activated cell sorting (MACS).

The invention also includes a non-human double transgenic animal comprising a first and a second bacterial artificial chromosome transgene, wherein the first bacterial artificial chromosome comprises a GLT-1 genomic DNA operatively linked to a first reporter gene, and wherein the second bacterial artificial chromosome comprises a GLAST genomic DNA operatively linked to a second reporter gene. In another embodiment, the invention includes a cell isolated from the non-human double transgenic animal.

Other features and advantages of the invention will be apparent from the following detailed description and claims.

### ABBREVIATIONS AND SHORT FORMS

The following abbreviations and short forms are used in this specification.

“BAC” means bacterial artificial chromosome.

“EAA” means excitatory amino acids.

“EAAT” means excitatory amino acids transporter.

“ELISA” means enzyme-linked immunosorbent assay.

“GFAP” means glial fibrillary acidic protein.

“PCR” means polymerase chain reaction.

“PND” means postnatal day.

“RT-PCR” means reverse transcription PCR.

### DEFINITIONS

The definitions used in this application are for illustrative purposes and do not limit the scope of the invention.

The articles “a” and “an” are used herein to refer to one or to more than one (e.g., to at least one) of the grammatical object of the article. By way of example, “an element” means one element or more than one element.

As used herein, each “amino acid” is represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change a peptide's circulating half life without adversely affecting activity of the peptide. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

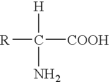

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an amino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino—and carboxy-terminal groups, although not specifically shown, will be understood to be in the form they would assume at physiologic pH values, unless otherwise specified.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

The term “complementary to” is used herein to mean that the subject sequence is homologous to all or a portion of a reference polynucleotide sequence. For illustration, the nucleotide sequence “TATAC” corresponds to a reference sequence “TATAC” and is complementary to a reference sequence “GTATA”.

The term “expression,” as used with respect to a glutamate transporter mRNA, refers to transcription of a glutamate transporter nucleic acid sequence, resulting in synthesis of a glutamate transporter mRNA. “Expression,” as used with respect to a glutamate transporter protein, refers to translation of a glutamate transporter mRNA, resulting in synthesis of a glutamate transporter protein.

As used herein, the term “fragment,” as applied to a nucleic acid, refers to a subsequence of a larger nucleic acid. A “fragment” of a nucleic acid can be at least about 20 nucleotides in length; for example, at least about 50 nucleotides to about 100 nucleotides; preferably at least about 100 to about 500 nucleotides, more preferably at least about 500 to about 1000 nucleotides, even more preferably at least about 1000 nucleotides to about 1500 nucleotides; particularly, preferably at least about 1500 nucleotides to about 2500 nucleotides; most preferably at least about 2500 nucleotides.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; more preferably at least about 100 amino acids in length, even more preferably at least about 200 amino acids in length, particularly preferably at least about 300 amino acids in length, and most preferably at least about 400 amino acids in length.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in any cell such as a prokaryotic cell, a virus, or a eukaryotic cell (including transgenic mammals); (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into any expression vector.

As used herein, “gene products” include any product that is produced in the course of the transcription, reverse-transcription, polymerization, translation, post-translation and/or expression of a gene. Gene products include, but are not limited to, proteins, polypeptides, peptides, peptide fragments, or polynucleotide molecules.

As used herein, the term “GLT-1” (or alternatively, “GLT1”) refers to the rodent astroglial glutamate transporter 2 gene. The mouse GLT-1 cDNA sequence is described in GenBank Accession Nos. AB007810, AB007812, and AB007811, as well as in Utsunomiya-Tate et al. (1997, FEBS Lett. 416:312-316). As used herein, the term “EAAT2” refers to the human astroglial glutamate transporter 2 gene. See, e.g., U.S. Pat. No. 5,658,782 which discloses the human EAAT2 cDNA sequence.

As used herein, the term “GLAST” refers to the rodent astroglial glutamate transporter 1 gene. The mouse GLAST cDNA sequence is described in GenBank Accession No. NM_{—}148938. As used herein, the term “EAAT1” refers to the human astroglial glutamate transporter 1 gene. See, e.g., U.S. Pat. No. 5,658,782 which discloses the human EAAT1 cDNA sequence.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, an isolated GLT-1 or GLAST nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

The term “mammal” as used herein refers to any non-human mammal. Such mammals are, for example, rodents, non-human primates, sheep, dogs, cows, and pigs. The preferred non-human mammals are selected from the rodent family including rat and mouse, more preferably mouse. A “transgenic mammal” as used herein refers to an mammal containing one or more cells bearing genetic information, received, directly or indirectly, by deliberate genetic manipulation at the subcellular level, such as by microinjection or transfection with recombinant DNA, or infection with recombinant virus. The term “transgenic mammal” also refers to a transgenic mammal in which the genetic information was introduced into a germ line cell, thereby conferring the ability to transfer the information to offspring. If such offspring in fact possess the transgene, they also are transgenic mammals.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant” as used herein, refers to either a nucleic acid or protein comprising a mutation.

As used herein, “neurological disorders” refers to diseases and disorders of the nervous system including, but are not limited to, amyotrophic lateral sclerosis (ALS), trinucleotide repeat expansion disorders (e.g., Huntington's disease (HD), spinal and bulbar muscular atrophy, spinocerebellar ataxia types 1, 2, 6, and 7, dentatorubropallidoluysian atrophy, and Machado-Joseph disease), α-synucleinopathies (e.g., Parkinson's disease (PD), dementia with Lewy bodies (DLB), and multiple system atrophy (MSA)), multiple sclerosis (MS), Alzheimer's disease, brain tumors (e.g., glioblastoma), stroke/ischemia, cerebrovascular disease, epilepsy (e.g., temporal lobe epilepsy), HIV-associated dementia, Korsakoff's disease, pain, headaches (e.g., migraine headaches), Pick's disease, progressive supranuclear palsy, Creutzfeldt-Jakob disease, Bell's Palsy, aphasia, sleep disorders, glaucoma, and Meniere's disease.

A “nucleic acid molecule” is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “oligonucleotide” typically refers to short polynucleotides of about 50 nucleotides or less in length. It will be understood that when a nucleotide sequence is represented herein by a DNA sequence (e.g., A, T, G, and C), this also includes the corresponding RNA sequence (e.g., a, u, g, c) in which “u” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids which can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit passive immunity.

“Pharmaceutically acceptable” means physiologically tolerable, for either human or veterinary applications.

As used herein, “pharmaceutical compositions” include formulations for human and veterinary use.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “promoter” refers to a region of a DNA sequence active in the initiation and regulation of the expression of a structural gene. This sequence of DNA, usually, but not necessarily exclusively, is upstream from the coding sequence of a structural gene, controls the expression of the coding region by providing the recognition for RNA polymerase and/or other elements required for transcription to start at the correct site.

As used herein, the term “psychiatric disorder” refers to diseases and disorders of the mind, and includes diseases and disorders listed in the Diagnostic and Statistical Manual of Mental Disorders—Fourth Edition (DSM-IV), published by the American Psychiatric Association, Washington D.C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

A “sample,” as used herein, refers to a biological sample from a subject, including normal tissue samples, blood, saliva, feces, or urine. A sample can also be any other source of material obtained from a subject which contains a compound or cells of interest.

A “subject,” as used herein, can be a human or non-human mammal. Non-human mammals include, for example, livestock and pets, such as ovine, bovine, porcine, canine, feline and murine mammals, as well as reptiles, birds and fish. Preferably, the subject is a human.

“Substantially purified” refers to a peptide or nucleic acid sequence which is substantially homogenous in character due to the removal of other compounds (e.g., other peptides, nucleic acids, carbohydrates, lipids) or other cells originally present. “Substantially purified” is not meant to exclude artificial or synthetic mixtures with other compounds, or the presence of impurities which do not interfere with biological activity, and which may be present, for example, due to incomplete purification, addition of stabilizers, or formulation into a pharmaceutically acceptable preparation.

As used herein, a “subunit” of a nucleic acid molecule is a nucleotide, and a “subunit” of a polypeptide is an amino acid.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

### DESCRIPTION OF THE FIGURES

For the purpose of illustrating the invention, there are depicted in the drawings certain embodiments of the invention. However, the invention is not limited to the precise arrangements and instrumentalities of the embodiments depicted in the drawings.

FIG. 1 depicts an exemplary schematic of the BAC modification steps for generating BAC transgenic constructs of the present invention. The BAC modification system of the invention comprises a two step homologous recombination process. First, a co-integration step, which results in a complete integration of a shuttle vector into the BAC, and a resolution step, which results in excision and subsequent loss of the shuttle vector and precise placement of the intended modification in the chosen site in the BAC.

FIG. 2 depicts a schematic of the modifications made to the GLAST BAC construct. The GLAST BAC construct is linked to the DsRed reporter.

FIG. 3 depicts a schematic of the modifications made to the GLT-1 BAC construct. The GLT-1 BAC construct is linked to the eGFP reporter.

FIG. 4, comprising FIGS. 4A through 4D, is a series of images depicting the validation of GLAST and GLT-1 BAC promoter reporter mice. FIG. 4A, comprising FIGS. 4A-1 through 4A-3, is a series of images of a brain from an adult GLAST-BAC-DsRed mouse including a bright field image (4A-1), DsRed fluorescence from the same brain (4A-2), and a close-up view from the cerebellum of the same brain (4A-3). FIG. 4B, comprising FIGS. 4B-1 through 4B-2, is a series of images depicting of a brain from an adult GLT-1-BAC-eGFP mouse including a bright field image (4B-1) and eGFP fluorescence from the same brain (4B-2). FIGS. 4C and 4D are Western blot images demonstrating no difference in GLAST or GLT-1 protein expression between transgenic reporter lines and wild type mice; 593 is a strong GLAST reporter line; 573 is a weak GLAST reporter line; 335 is a strong GLT-1 reporter line; and 356 is a weak GLT-1 reporter line.

FIG. 5, comprising FIGS. 5A and 5B, is a series of images depicting developmental changes in BAC transgenic mice. FIG. 5A depicts GLAST promoter activity in different areas of the brain at various postnatal days (PND). FIG. 5B is a series of images depicting developmental changes in GLT-1 promoter activity in different areas of the brain at various postnatal days (PND).

FIG. 6, comprising FIGS. 6A through 6H, is a series of images highlighting the different expression patterns of the GLAST and GLT-1 transporters at postnatal days 1 and 24 in double GLAST-DsRed/GLT-1-eGFP BAC transgenic mice. FIGS. 6A and 6B depict the cerebellum (Cblm). FIGS. 6C and 6D depict the cerebral cortex (Ctx). FIGS. 6E and 6F depict the hippocampus (Hipp). FIGS. 6G and 6H depict the spinal cord (SC). Scale bars=300 μm.

FIG. 7, comprising FIGS. 7A through 7E, is a series of images demonstrating that GLAST and GLT-1 promoters are active in some distinct subsets of cells in the hippocampus and in spinal cord at postnatal day 24. FIG. 7A demonstrates that GFAP protein, GLAST promoter activation, and GLT-1 promoter activation in the hippocampus dentate gyrus overlap in the radial glia, but not always in the granule cell layer. FIG. 7B demonstrates that in the hippocampus CA3 region, many GFAP+astrocytes exhibit GLAST and GLT-1 promoter activation. However, there are also some non-astrocytic GLAST expressing cells. FIG. 7C-7E depicts spinal cord of double transgenic mice: C) ventral white column; D) dorsal white column; E) Gray matter (Gm)/white matter (Wm) junction stained with GFAP (scale bars=50 μm). As shown in FIGS. 7C and 7D, the GLAST and GLT-1 promoters are active in distinct subsets of cells. However, as shown in FIG. 7E, the GLAST promoter-active cells are not GFAP+.

FIG. 8, comprising FIGS. 8A through 8J, is a series of images depicting GLT-1 and GLAST promoter activity in various cell populations. FIG. 8A shows that GLT-1 and GLAST promoters are both active in the radial glia of the dentate gyrus. FIG. 8B shows that a majority of GLAST promoter-active cells were negative for both GLT-1 promoter expression and neuronal markers (i.e. NeuN) in the corpus callosum. FIG. 8C shows that these GLAST promoter-active cells in the corpus callosum are not oligodendroglia. FIG. 8D shows that a population of cells in the cortex are non-GLT-1 promoter activated GLAST cells (i.e. GLAST promoter is active and GLT-1 promoter is not active). These small round cells appear to be neurons or neuron progenitors based on co-staining with NeuN. FIG. 8E shows that a large number of GLAST-promoter active cells in the cortex are oligodendrocytes. FIG. 8F shows that a majority of GLAST promoter-active cells in the striatum are not astrocytes, whereas FIG. 8G shows that many GLAST promoter-active cells are oligodendrocytes. In spinal cord, activation of the GLT-1 and GLAST promoters was completely independent, and also completely non-overlapping with neurons, as shown in FIG. 8H. The vast majority of gray matter astrocytes demonstrated GLT-1 promoter activation. The GLAST promoter-active cells were pronounced in the white matter tracts, and many of these cells appeared to be oligodendroglia (FIG. 8I), but not neurons (FIG. 8H) or microglia (FIG. 8J). Scale bars=50 μm.

### DETAILED DESCRIPTION OF THE INVENTION

The invention includes a transgenic, non-human mammal, and cells isolated from the mammal for identifying a candidate compound useful in the treatment of a neurological disorder and/or a psychiatric disorder.

The present invention is based, at least in part, on the generation of novel non-human transgenic mammals and cells for use in identifying compounds useful for the treatment of neurological and/or psychiatric disorders. The mammals and cells of the present invention contain transgenes derived from bacterial artificial chromosomes (BAC) comprising a glutamate transporter locus and a reporter gene operatively linked to the glutamate transporter promoter. The BAC transgene used in the invention is preferably derived from the same species as the non-human transgenic mammal, and preferably comprises the natural glutamate transporter promoter and regulatory elements. Without wishing to be bound by any particular theory, the use of these same-species BAC-reporter transgenes ensures that the reporter genes are regulated in vivo in the same manner as the natural glutamate transporter genes.

The invention also relates to the production of a transgenic non-human mammal containing within its genome a reporter gene operatively linked to a promoter sequence derived from a bacterial artificial chromosome (BAC) clone, wherein the BAC clone comprises a genomic sequence comprising a locus selected from the group consisting of the GLT-1 locus and the GLAST locus. Preferably, the BAC clone is selected from the group consisting of RPCI-23-361-H22 and BAC clone RPCI-24-287G11.

In another aspect, the present invention provides a non-human, transgenic mammal, preferably a transgenic rodent, more preferably a transgenic mouse, useful for identifying and providing cells comprising a glutamate transporter promoter operatively linked to a reporter gene. In accordance with the present invention, the cells are isolated from the transgenic mammal and cultured for use in screening methods for identifying a candidate compound useful in treating a neurological and/or psychiatric disorder. Preferably, the cells are derived from the central nervous system (CNS) of the transgenic mammal. In addition, the test compound can be administered to the transgenic mammal and the test compound can be assayed for the ability of the compound to modulate the expression of the reporter gene, thereby providing an indication of the activity of the glutamate transporter promoter (i.e. GLT-1 and GLAST).

In another embodiment, a nucleic acid sequence derived from a BAC clone comprising a glutamate transporter locus and a reporter gene operatively linked to a glutamate transporter promoter is incorporated into the genome of the transgenic mammal and cells isolated therefrom. Preferably, the glutamate transporter promoter is selected from the group consisting of the GLT-1 promoter and the GLAST promoter. The reporter gene serves as a detectable marker that permits identification of cells that are expressing such a marker from cells that are not expressing the reporter gene marker. In some instances, the reporter gene can also serve for isolating cells that are expressing such a marker from cells that are not expressing the reporter gene marker.

The nucleotide sequences (cDNA) used herein were cloned using standard molecular biology techniques (Maniatus et al., Molecular Cloning: A Laboratory Manual, Cold Spring Harbor Laboratory (1982); Ausubel et al., Current Protocols in Molecular Biology, John Wiley and Sons, Volume 2 (1991)) based on sequences available in the public domain (e.g., GenBank).

The construct may also comprise selected nucleic acid regions associated with the transgene (as by fusion therewith) for mediation of, for example, its introduction into the target genome, its expression loci in the transgenic mammal, on/off external regulation of transgene expression, and other desired features, as generally known in the art.

In another aspect, the present invention relates to a DNA construct comprising the transgene. Such a construct is an expression vector, preferably a plasmid which allows for preparation of large amounts of the transgene. In such a plasmid, the transgene is flanked by restriction sites and preferably comprises an origin of replication. Such a construct may be made by cloning the promoter sequence into a vector comprising the reporter gene sequence, or by cloning the reporter gene sequence into a vector comprising the promoter sequence, using conventional recombinant techniques. The DNA sequence encoding the promoter is incorporated into the construct in appropriate frame with the reporter gene sequence such that induction of the promoter causes expression of the reporter gene.

In another aspect, the present invention relates to a zygote or embryonic stem cell whose genome comprises the transgene. A DNA construct which comprises the transgene may be integrated into the genome of the transgenic mammal by any standard method such as those described in Hogan et al., “Manipulating the Mouse Embryo”, Cold Spring Harbor Laboratory Press, 1986; Kraemer et al., “Genetic Manipulation of the Early Mammalian Embryo”, Cold Spring harbor Laboratory Press, 1985; Wagner et al., U.S. Pat. No. 4,873,191, Krimpenfort et al U.S. Pat. No. 5,175,384 and Krimpenfort et al., Biotechnology, 9: 88 (1991), all of which are incorporated herein by reference. Preferably, the DNA fragment is microinjected into pronuclei of zygotes of non-human mammalian mammals, such as mice, rabbits, cats, dogs, or larger domestic or farm mammals, such as pigs. These injected embryos are transplanted to the oviduts or uteri of pseudopregnant females from which founder mammals are obtained. The founder mammals (Fo), are transgenic (heterozygous) and can be mated with non-transgenic mammals of the same species to obtain F1 non-transgenic and transgenic offspring at a ratio of 1:1. A heterozygote mammal from one line of transgenic mammals may be crossed with a heterozygote mammal from a different line of transgenic mammals to produce mammals that are heterozygous at two loci. Mammals whose genome comprises the transgene are identified by standard techniques such as polymerase chain reaction or Southern assays or otherwise methods disclosed herein.

The heterozygote offspring in the F1 generation or F2 generation exhibit reporter expression. Accordingly, the heterozygous transgenic mammals are useful tools for screening candidate agents capable of modulating the promoter activity. Such transgenic mammals are also useful for studying developmental changes in the promoter activity.

In another aspect, the present invention provides a method for identifying compounds which are effective at modulating a glutamate transporter promoter activity. The method comprises administering the candidate compound to the transgenic mammal and monitoring the promoter activity by way of measuring the reporter expression phenotype. Preferably, varying doses of the candidate compound are introduced into the separate transgenic mammals intracerebrally or by conventional modes of injection, such as for example, by intravenous injection or intraperitoneal injection.

The present invention also relates to an isolated cell or a cell line derived from the transgenic mammal whose genome comprises the glutamate transporter promoter operatively linked to a reporter gene. Such cells are obtained from the appropriate area of the CNS of such a transgenic mammal using conventional methods known in the art. The cells of the present invention are useful for therapy in patients suffering from a neurological disorder and/or psychiatric disorder.

In one embodiment, the cell isolated from the transgenic mammal of the invention is selected from the group consisting of mGLT1-BAC-eGFP-GRiP1, mGLAST-BAC-DsRed-GRiP1, and mGLT1-BAC-eGFP/mGLAST-BAC-DsRed-GRiP1.

In another aspect, the present invention comprises a method of obtaining a cell from the transgenic mammal. The cells can be isolated from the transgenic mammal using standard techniques. For example, the cells can be isolated from the transgenic mammal or a population of cells in a sample derived from the transgenic mammal using the proper detection method, either by drug/metabolite selection, fluorescence activated cell sorting, immunodetective “panning,” or immunohistochemistry. These cells could then be propagated for cell based therapy in patients suffering from a neurological and/or psychiatric disorder.

In one embodiment, the cells isolated from the transgenic mammal are obtained from the CNS of the mammal. The cells isolated from the CNS are preferably selected from the group consisting of astrocytes, oligodendrocytes, oligodendrocyte precursor cells, endothelial cells, microglia, neurons, ependymal cells, radial glia, Bergmann glia, and tanyctes.

In another embodiment, the cells isolated from the transgenic mammal are obtained from a sample other than a CNS tissue sample. The isolated cell can be selected from the group consisting of an embryonic stem cell, a bone marrow cell, or other types of cells, which may be used as pools of candidate cells to select for cells comprising a BAC transgene, wherein the BAC comprises genomic DNA comprising a locus selected from the group consisting of the GLT-1 locus and the GLAST locus. Preferably, the cells have a detectable level of reporter gene expression.

The non-human transgenic mammals and cells of the present invention provide novel diagnostic targets and means for identifying therapeutic agents for neurological and/or psychiatric disorders. The neurological disorders that can be treated in accordance with the present invention include disorders that have been reported to be associated with excitotoxicity and/or glutamate neurotransmission. Particularly included are specified neurological disorders affecting motor neuron function.

As noted, neurological and/or psychiatric disorders of specific interest include those associated with abnormal release or removal of excitotoxic amino acids such as glutamate. Several CNS neuron types are especially adversely affected by excitotoxic glutamate. See e.g., Choi, D. W. (1988, Neuron 1: 623; and references cited therein). Particularly preferred neurological disorders include AD, HD, PD with ALS being most preferred.

In addition, the methods and compositions of the present invention provide for selection of therapeutic agents for the modulation of normal glutamate neurotransmission associated with brain functions such as learning and memory. For example, the therapeutic agents identified using the methods and compositions described herein may be used to enhance learning and memory.

Promoter

Transcriptional regulation of protein expression is achieved through the interaction of positive and negative regulatory molecules with a gene's promoter, an ill-defined region of DNA generally, but not necessarily exclusively, upstream of the start codon. That is, a promoter is a region of genomic DNA, usually found 5′ to an mRNA transcription start site. Promoters are involved in regulating the timing and level of mRNA transcription and contain, for example, binding sites for cellular proteins such as RNA polymerase and other transcription factors. Because of technical and practical issues, cloned promoters are typically short fragments of DNA immediately upstream of the initiating ATG.

A promoter may also contain large regions of DNA both before, within, and after a gene. BAC DNA which includes the entire gene plus significant lengths of DNA both before and after may more accurately replicate wild type promoter activity. In fact, a fragment of the promoter may not always recapitulate natural In vivo gene regulation.

A promoter region can contain additional promoter elements, i.e., enhancers, that regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. Depending on the promoter, individual elements can function either co-operatively or independently to activate transcription.

A method of assessing the activity of a promoter is to operably link the promoter to a gene product such a reporter gene. Reporter genes are used for identifying and evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

As discussed elsewhere herein, suitable reporter genes may include genes encoding luciferase, beta-galactosidase, chloramphenicol acetyl transferase, secreted alkaline phosphatase, or fluorescent protein gene (see, e.g., Ui-Tei et al., 2000 FEBS Lett. 479:79-82). In general, a promoter region may be linked to a reporter gene and used to evaluate the promoter activity and the ability of agents to modulate promoter-driven transcription.

One aspect of the invention relates to bacterial artificial chromosomes (BAC) clone comprising the desired promoter sequence. BACs are advantageous over other sources of promoter sequence because BAC DNA includes the entire gene and additional sequences that may provide a more accurate wild type promoter. BACs are easy to manipulate because they are stable in their hosts. In support of the functional analysis of genes, the BACs are very useful for making transgenic animals with segments of heterologous DNAs.

Isolated Nucleic Acid Molecules

One aspect of the invention pertains to isolated nucleic acid molecules, including BACs, that comprise a glutamate transporter and a reporter gene or biologically active portions thereof, as well as nucleic acid fragments sufficient for use as hybridization probes to identify the glutamate transporter promoter containing nucleic acid molecules and fragments. The invention also includes PCR primers for the amplification/mutation of the glutamate transporter promoter and reporter gene.

In general, optimal practice of the present invention can be achieved by using recognized manipulations. For example, techniques for isolating mRNA, methods for making and screening cDNA libraries, purifying and analyzing nucleic acids, methods for making recombinant vector DNA, cleaving DNA with restriction enzymes, ligating DNA, introducing DNA into host cells by stable or transient means, culturing the host cells, methods for isolating and purifying polypeptides and making antibodies are generally known in the field. See Sambrook et al., supra.

A nucleic acid molecule used in the present invention can be isolated using standard molecular biology techniques and the sequence information provided herein. Using all or, a portion of the nucleic acid sequence of GLT-1 or GLAST corresponding to the promoter region, nucleic acid molecules can be isolated using standard hybridization and cloning techniques (e.g., as described in Sambrook et al., supra).

Moreover, a nucleic acid molecule encompassing all or a portion of GLT-1 or GLAST promoter region can be obtained by the polymerase chain reaction (PCR) using synthetic oligonucleotide primers designed based upon the sequences of GLT-1 or GLAST promoter region.

A nucleic acid used in the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to GLT-1 or GLAST nucleotide sequences can be prepared by standard synthetic techniques, e.g., using an automated DNA synthesizer.

In still another embodiment, an isolated nucleic acid molecule used in the invention comprises a nucleic acid molecule which is a complement of the nucleotide sequence of GLT-1 or GLAST, or a portion of any of these nucleotide sequences. A nucleic acid molecule which is complementary to the nucleotide sequence of GLT-1 or GLAST is one which is sufficiently complementary to the nucleotide sequence of GLT-1 or GLAST such that it can hybridize to the nucleotide sequence of GLT-1 or GLAST, thereby forming a stable duplex. The term “complementary” or like term refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99 to about 100%. Complementary polynucleotide sequences can be identified by a variety of approaches including use of well-known computer algorithms and software.

In still another embodiment, an isolated nucleic acid molecule used in the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to the nucleotide sequence of GLT-1 or GLAST (e.g., to the entire length of the nucleotide sequence), or a portion or complement of any of these nucleotide sequences. In one embodiment, a nucleic acid molecule used in the present invention comprises a nucleotide sequence which comprises part or all of GLT-1 or GLAST, or a complement thereof, and which is at least (or no greater than) 25, 30, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1250, 1300, 1350, 1400, 1450, 1500, 1550, 1600, 1650, 1700, 1750, 1800, 1850, 1900, 1950, 1994, 2000, 2050, 2073, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3441, 3450, 3500, 3550, 3600, 3650, 3700, 3750, 3800, 3841, 3850, 3900, 3950; 4000, 4050, 4100, 4150, 4200, 4250, 4300, 4350, 4400, 4450, 4500, 4550, 4600, 4650 or more nucleotides (e.g., contiguous nucleotides) in length.

To determine the percent identity of two nucleic acid or amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to a nucleotide sequence having 100 nucleotides, at least 30, preferably at least 40, more preferably at least 50, even more preferably at least 60, and even more preferably at least 70, 80, or 90 nucleotides are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970, J. Mol. Biol. 48:444-453) algorithm which has been incorporated into the GAP program in the GCG software package (available at online through the Genetics Computer Group), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at online through the Genetics Computer Group), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blosum 62 scoring matrix with a gap penalty of 12, a gap extend penalty of 4, and a frame shift gap penalty of 5.

In another embodiment, the percent identity between two amino acid or nucleotide sequences is determined using the algorithm of Meyers and Miller (Comput. Appl. Biosci. 4:11-17 (1988)) which has been incorporated into the ALIGN program (version 2.0 or version 2.0U), using a PAM120 weight residue table, a gap length penalty of 12 and a gap penalty of 4.

The nucleic acid and protein sequences used in the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to GLT-1 or GLAST nucleic acid molecules of the invention. BLAST protein searches can be performed with the XBLAST program, score=50, wordlength=3 to obtain amino acid sequences homologous to glutamate transporter protein molecules used in the invention. To obtain gapped alignments for comparison purposes, Gapped BLAST can be utilized as described in Altschul et al. (1997, Nucleic Acids Res. 25:3389-3402). When utilizing BLAST and Gapped BLAST programs, the default parameters of the respective programs (e.g., XBLAST and NBLAST) can be used. See the website for the National Center for Biotechnology Information.

The nucleic acid molecule used in the invention can comprise only a portion of the nucleic acid sequence of GLT-1 or GLAST promoter region. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence the GLT-1 and/or GLAST promoter region, or a complement thereof.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the GLT-1 or GLAST nucleotide sequences can be used to detect (e.g., specifically detect) genomic sequences. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of a GLT-1 or GLAST sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which mis-express a protein whose expression is controlled by a GLT-1 or GLAST, such as by measuring a level of promoter activity in a sample of cells from a subject, e.g., determining whether a genomic promoter has been mutated or deleted.

In another embodiment, nucleic acid molecules used in the invention can comprise variants of the sequences disclosed herein. Nucleic acid variants can be naturally occurring, such as allelic variants (same locus), homologues (different locus), and orthologues (different organism, e.g., rat) or can be non-naturally occurring. Non-naturally occurring variants can be made by mutagenesis techniques, including those applied to polynucleotides, cells, or organisms. The variants can contain nucleotide substitutions, deletions, inversions and insertions.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4 × sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3× SSC, at about 65-70° C. A preferred, non-limiting example of reduced stringency hybridization conditions includes hybridization in 4×SSC at about 50-60° C. (or alternatively hybridization in 6×SSC plus 50% formamide at about 40-45° C.) followed by one or more washes in 2X SSC, at about 50-60° C. Ranges intermediate to the above-recited values, e.g., at 65-70° C. or at 42-50° C. are also intended to be encompassed by the present invention. SSPE (1× SSPE is 0.15M NaCl, 10 mM NaH₂PO₄, and 1.25 mM EDTA, pH 7.4) can be substituted for SSC (1×SSC is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N) where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non- limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

Preferably, an isolated nucleic acid molecule used in the invention that hybridizes under stringent conditions to the sequence of GLT-1 or GLAST corresponds to a naturally-occurring nucleic acid molecule. As used herein, a “naturally-occurring” nucleic acid molecule refers to an RNA or DNA molecule having a nucleotide sequence that occurs in nature.

BACs, Recombinant Expression Vectors, and Host Cells

Another aspect of the invention pertains to vectors, for example recombinant expression vectors, containing a GLT-1 or GLAST nucleic acid molecule. A vector refers to a nucleic acid molecule capable of transporting another nucleic acid to which it has been linked. One type of vector is a plasmid, which refers to a circular double stranded DNA loop into which additional DNA segments can be ligated. Another type of vector is a viral vector, wherein additional DNA segments can be ligated into the viral genome.

A preferred type of vector for use in the present invention is a bacterial artificial chromosome (BAC). BACs are vectors, based on a naturally occurring F-factor plasmid found in the bacterium E. coli, which are used to clone DNA fragments (100- to 300-kb insert size; average, 150 kb) in E. coli cells. BACs are often used as vectors to produce genomic libraries.

BACs have several advantages over the traditional large DNA cloning system, the yeast artificial chromosomes (YACs). These include large carrying capacity (˜100-300 kb), high clonal stability, low rate of chimerism, and the ease with which they can be handled. BACs have served as the primary source of archived genomic DNA for a variety of genome mapping and sequencing projects. Indeed, physical contigs are now available for the entire human genome and the mouse genome, as well as a variety of other experimental organisms. An advantage of using a BAC system is that BAC DNA contains the entire gene plus addition sequences that may provide a more accurate replicate of the corresponding wild type gene. For example, a glutamate transporter promoter derived from a BAC clone provides a better representation of the corresponding wild type promoter activity.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked (i.e. expression vectors). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g. polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). In a preferred embodiment, the regulatory sequences in the expression vectors of the invention are derived from the GLT-1 or GLAST promoter of the invention, and the nucleic acid sequence to be expressed is a reporter gene, as described elsewhere herein. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

As used herein a “reporter” or a “reporter gene” refers to a nucleic acid molecule encoding a detectable marker. Preferred reporter genes include luciferase (e.g., firefly luciferase or Renilla luciferase), β-galactosidase, chloramphenicol acetyl transferase (CAT), and a fluorescent protein (e.g., green fluorescent, protein, red fluorescent protein (DsRed), yellow fluorescent protein, blue fluorescent protein, cyan fluorescent protein, or variants thereof, including enhanced variants such as enhanced GFP (eGFP)). Reporter genes must be detectable by a reporter assay. Reporter assays can measure the level of reporter gene expression or activity by any number of means, including measuring the level of reporter mRNA, the level of reporter protein, or the amount of reporter protein activity.

Methods for measuring mRNA levels are well-known in the art and include, but are not limited to, Northern blotting, RT-PCR, primer extension, and nuclease protection assays. Methods for measuring reporter protein levels are also well-known in the art and include, but are not limited to, Western blotting, ELISA, and RIA assays. Reporter activity assays are still further well-known in the art, and include luciferase assays, β-galactosidase, and chloramphenicol acetyl transferase (CAT) assays. Fluorescent protein activity can be measured by detecting fluorescence of the fluorescent protein. Therefore, GLT-1 and/or GLAST promoter activity can be measured using any method of detecting the reporter activity described elsewhere herein.

The recombinant expression vectors of the invention are preferably designed for expression in eukaryotic cells (e.g., mammalian cells). Alternatively, the recombinant expression vector can be transcribed and translated in vitro.

Another aspect of the invention pertains to host cells into which a BAC-reporter nucleic acid molecule of the invention is introduced, e.g., a GLT-1 or GLAST BAC-reporter nucleic acid molecule within a vector (e.g., a recombinant expression vector) or a GLT-1 or GLAST BAC-reporter nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a vector containing a GLT-1 or GLAST BAC-reporter nucleic acid molecule can be propagated and/or expressed in bacterial cells such as E. coli, insect cells, yeast or mammalian cells (such as Chinese hamster ovary cells (CHO), COS cells (e.g., COS7 cells), C6 glioma cells, HEK 293T cells, or neurons). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a GLT-1 or GLAST BAC-reporter nucleic acid molecule or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an mRNA or protein (e.g., a reporter mRNA or protein) encoded by the nucleic acid molecule operatively linked to the GLT-1 or GLAST promoter. Accordingly, the invention further provides methods for producing an mRNA or protein using the host cells of the invention. In one embodiment, the method comprises culturing the host cell of the invention in a suitable medium such that mRNA and/or protein encoded by the operatively linked nucleic acid molecule is produced. In another embodiment, the method further comprises isolating the mRNA and/or protein from the medium or the host cell.

Transgenic Mammal and Cells Derived therefrom

The host cells of the invention can also be used to produce non-human transgenic mammals. For example, in one embodiment, a host cell of the invention is a fertilized oocyte or an embryonic stem cell into which GLT-1 or GLAST BAC-reporter nucleic acid molecule sequences have been introduced. Such host cells can then be used to create non-human transgenic mammals in which exogenous GLT-1 or GLAST BAC-reporter sequences have been introduced into their genome or homologous recombinant mammals in which endogenous promoter sequences may or may not have been altered. Such mammals are useful for studying the function and/or activity of the desired promoter and for identifying and/or evaluating modulators of the promoter activity (i.e. GLT-1 and GLAST). As used herein, a “transgenic mammal” is a non-human mammal, preferably a mammal, more preferably a rodent such as a rat or mouse, in which one or more of the cells of the mammal includes a transgene. Other examples of transgenic mammals include non-human primates, sheep, dogs, cows, goats, chickens, amphibians, and the like. A transgene is exogenous DNA which is integrated into the genome of a cell from which a transgenic mammal develops and which remains in the genome of the mature mammal, thereby directing the expression of an encoded gene product in one or more cell types or tissues of the transgenic mammal.

A transgenic mammal of the invention can be created by introducing a GLT-1 or GLAST BAC-reporter nucleic acid molecule into the male pronuclei of a fertilized oocyte, e.g., by microinjection or retroviral infection, and allowing the oocyte to develop in a pseudopregnant female foster mammal. Methods for generating transgenic mammals via embryo manipulation and microinjection, particularly mammals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo (Cold Spring Harbor Laboratory Press, Cold Spring Harbor, N.Y., 1986). Similar methods are used for production of other transgenic mammals. A transgenic founder mammal can be identified based upon the presence of the reporter transgene in its genome and/or expression of the reporter gene in tissues or cells of the mammals. A transgenic founder mammal can then be used to breed additional mammals carrying the transgene. Moreover, transgenic mammals carrying a transgene containing GLT-1 or GLAST BAC-reporter can further be bred to other transgenic mammals carrying other transgenes.

In a preferred embodiment, transgenic mammals of the invention are produced using bacterial artificial chromosomes (BACs) containing the GLT-1 or GLAST locus. In a preferred embodiment, the GLT-1 or GLAST locus is derived from the same locus as the transgenic mammal in which it will be inserted. In another preferred embodiment, a BAC containing the GLT-1 or GLAST locus is modified such that it contains a reporter gene under the control of the GLT-1 or GLAST promoter. An exemplary method for BAC modification is described by Gong, S. et al. (Gong, S. et al., 2002 Genome Res. 12:1992-1998), and is also described elsewhere herein in the Examples section. The final modified BAC is linearized and injected into mouse pronuclei for generation of a transgenic reporter mice.

BAC clones containing the GLT-1 and GLAST loci may be identified by screening a BAC library with a GLT-1 or GLAST nucleic acid probe (e.g., a GLT-1 or GLAST cDNA probe). Numerous BAC libraries are known in the art, and are available through the BACPAC Resource Center (BPRC) at Children's Hospital Oakland Research Institute (CHORI), 747 52nd St. Oakland, Calif. 94609, USA. In a preferred embodiment, the BAC clone RPCI-23-361H22 (also referred to as RP23-361H22) is used for construction of GLT-1 transgenic mice. In a further preferred embodiment, the BAC clone RPCI-24-287G1 I (also referred to as RP24-287G1 1) is used for construction of GLAST transgenic mice. The GLAST BAC sequence is on mouse chromosome 15; the GLAST is gene slcl1a3 on mouse chromosome 15 at locus position 8425087-8425252 in Build 34.1. The GLT-1 BAC sequence is on mouse chromosome 2; the GLT-1 is gene slcla2 on mouse chromosome 2 at locus position 102411193-102482861 in Build 34.1.

Reporter genes may be operatively linked to the GLT-1 or GLAST promoters by flanking the reporter gene with GLT-1 or GLAST cDNA sequences, and using homologous recombination to insert the reporter gene into the GLT-1 or GLAST locus in the BAC.

BAC modification initially involves restoration of the competence for homologous recombination by introduction into the host cell of an enzyme or enzymes that can complement its deficiency for homologous recombination, and selection against freely replicating shuttle vector to identify cells in which the shuttle vector had co-integrated into the BAC by homologous recombination.

An aspect of the invention includes a BAC modification system that uses a two step homologous recombination process: a co-integration step, which results in complete integration of a shuttle vector into the BAC, and a resolution step, which results in excision and subsequent loss of the shuttle vector and precise placement of intended modification in the chosen site on the BAC. Such a two-step modification protocol is highly desirable for transgenic studies because it results in precise modification of the BAC without leaving any unwanted sequences behind.

In the first step, a shuttle vector carrying the recombination cassette is electroporated into the BAC host, and the correct co-integrates are selected. The co-integrates are identified using methods known in the art, for example through colony PCR, and the positive clones can be confirmed by Southern blot analysis. In the second step, resolved clones are selected and tested by known methods such as colony PCR, and the positive clones can be confirmed by Southern blot analysis. The modified BACs are fingerprinted by two or three different restriction digestions and are compared with that of the wild-type BACs to ensure that there are no rearrangements or deletions.

Clones of the non-human transgenic mammals described herein can also be produced according to the methods described in Wilmut et al. (1997, Nature 385:810-813) and PCT International Publication Nos. WO 97/07668 and WO 97/07669. In brief, a cell, e.g., a somatic cell, from the transgenic mammal can be isolated and induced to exit the growth cycle and enter G₀ phase. The quiescent cell can then be fused, e.g., through the use of electrical pulses, to an enucleated oocyte from a mammal of the same species from which the quiescent cell is isolated. The reconstructed oocyte is then cultured such that it develops to morula or blastocyte and then transferred to pseudopregnant female foster mammal. The offspring borne of this female foster mammal will be a clone of the mammal from which the cell, e.g., the somatic cell, is isolated.

Transgenic and homologous recombinant mammals of the invention can also be used to produce stable cell lines containing the GLT-1 and/or GLAST BAC-reporter transgenes. Such cell lines are useful because they can be made so that they do not overexpress the transgene (as may happen in transient transfection), and therefore more closely reflect the natural cellular environment of the transgene. Such cell lines may be produced by isolating the desired cells (e.g., fibroblasts or astroglial cells) from a transgenic or homologous recombinant mammal (e.g.,. a mouse) and culturing them using standard methods. In some embodiments primary (i.e., non-immortalized) cells are preferred, or the cells may be may be immortalized (e.g., by the addition of a gene such as SV40 large T antigen) in order to propagate them indefinitely in culture.

Transgenic Mammal and Cells Derived therefrom

Transgenic mice are important tools for in vivo genetic studies. Because the genomic DNA inserts in BACs are sufficiently large, BACs carry an entire transcription unit and its associated regulatory regions. Therefore, BACs can be used for a variety of functional studies that could not be accomplished by using conventional transgenic approaches. The ability to manipulate BAC constructs provide strategies for gene expression and function studies, as well as cell marking and isolation studies.

Besides containing more of the actual promoter DNA, BAC reporter mice offer the opportunity to visualize cells with lower promoter activation levels, which would be overwhelmed in many cases with in situ hybridization, or with immunohistochemistry using an antibody against a protein epitope. For example, GLT-1/EAAT2 has an extremely high level of expression in brain tissue (approx 1% of total protein) and demonstrates a strong neuropil staining using standard immunohistochemical techniques. Therefore, it is difficult to differentiate individual cellular patterns of expression where the transporter could also be playing an important role.

Another advantage that these mice provide is the ability to study living tissue using fluorescent reporters and electrophysiology. Thus, it is possible to identify and “patch clamp” specific cells to study functional expression levels. For instance, the relative contribution of the GLAST transporter to uptake of both synaptic and ambient glutamate can be examined. In addition, because mice with different colored reporters (GLT-1 promoter reporter is eGFP; GLAST reporter is DsRed) was constructed, it is possible to compare expression in specific cells. Furthermore, as relative eGFP levels have been shown to be indicative of relative promoter activation in promoter reporter mice, the GLT-1 BAC promoter reporter is helpful for identifying and developing potential pharmacological activators and inhibitors associated with glutamate.

The invention comprises a transgenic non-human mammal whose germ cells and somatic cells express a reporter gene under the regulation of a GLT-1 and/or GLAST promoter. Cells in which the GLT-1 and/or GLAST promoter are activated can be readily determined by the expression of the reporter gene, which can be measured by standard detection techniques known to those skilled in the art or otherwise disclosed herein. The invention further comprises non-human mammalian embryos carrying the reporter gene operatively linked to the glutamate transporter promoter capable of developing into viable transgenic mammals whose progeny carry the reporter gene after breeding forward by sexual reproduction.

The transgenic non-human mammals of the invention are characterized by the emission of light in tissues that contain an active GLT-1 and/or GLAST promoter. In a preferred embodiment of the invention the transgenic non-human mammals are utilized as a model or surrogate for human GLT-1 and/or GLAST promoter function for the identification and optimization of molecules and compounds that modulate the promoter activity. Molecules and compounds so identified can be used in the prevention and treatment of disorders associated with defective glutamate transporter promoter function including, but not limited to ALS, multiple sclerosis, stroke, Alzheimer's and the like. Thus, the invention provides an in vivo system to monitor the activity of the glutamate transporter promoter in different organs and tissues.

GLT-1 and/or GLAST promoter activity can be monitored using the transgenic mammals of the present invention. It was observed that GLAST promoter activity in a newborn mouse was highest in the cerebral cortex and periventricular progenitor proliferation zones within the brain. This activation in cortex is greatly reduced during development, whereas the GLAST promoter was observed to be highly active in radial glia of the hippocampal dentate gyrus and in Bergmann glia of the cerebellum in the adult mouse. The GLAST promoter reporter transgenic mice exhibited activity with respect to the GLAST promoter in both radial glia and in many astrocytes in the developing CNS, but was down regulated in most astrocytes as mammals matured. In contrast to a restricted pattern of expression in gray matter, widespread activation of the GLAST promoter was observed in oligodendrocytes in white matter throughout many (i.e., spinal cord and corpus callosum), but not all (i.e., cerebellum), CNS regions. GLAST promoter activity was not observed in a variety of non neuronal cells including, but not limited to, endothelial cells, NG2 positive cells, and microglia. In the adult CNS, the highest promoter activity was observed in radial glia, such as those located in the subgranular layer of the dentate gyrus.

With respect to GLT-1 promoter activity, histological analysis of GLT-1 promoter reporter mice revealed that GLT-1 promoter activity in the CNS was largely restricted to astrocytes, but was also observed to a lesser degree in CA3 pyramidal neurons.

The transgenic mammals of the invention also provide for a method of assessing the cellular expression patterns of GLT1 and/or GLAST in the same mammal. That is, the invention includes crossing a transgenic mammal comprising a reporter gene (i.e. DsRed) operatively linked to a GLAST promoter with a different transgenic mammal comprising a different reporter gene (i.e. eGFP) operatively linked to a GLT promoter, thereby creating a double transgenic mammal. An advantage of using a double transgenic mammal having different reporters (i.e. different colors), is that it is possible to compare expression of both promoters in specific cells. Also, whether it is a transgenic mammal or a double transgenic mammal, the GLT-1 and/or GLAST BAC promoter reporter is helpful for identifying and assessing potential pharmacological activators and inhibitors of promoter activity. The double transgenic mammal is also useful for identifying specific BAC promoter-expressing cells and analyzing transporter function in living slices, and to assess up- and down-regulation of transporter promoter activation in different pathological, physiological, and pharmaceutical paradigms.

The transgenic mammals of the invention can be used to assess both GLAST and GLT1 expression by way of measuring the expression of the distinct reporter genes. Based on the disclosure presented herein, there are some overlapping areas in the double transgenic mammal that GLAST and GLT1 promoter activity was observed. In addition, there were some distinct areas in the double transgenic mammal that GLT-1 and GLAST promoter activity was observed. For example, in the hippocampus, both promoters were active and prominent. However, in the spinal cord, there was little overlap between promoter activation of GLAST and GLT-1, with GLT1 expression widely present in the gray matter and GLAST promoter activity strongly present in white matter tracts (GLAST promoter activity was also present in the gray matter but not as predominant as compared with GLAST promoter activity in the white matter). Similar independent promoter activity of GLAST and GLT1 was seen in cortex, striatum and brainstem.

In addition to the observation that different areas of the transgenic mammal (i.e. different areas of the brain) exhibited distinct GLT-1 and GLAST promoter activity patterns, it was also observed that GLT-1 and GLAST promoter activity patterns was exhibited in distinct cell populations. That is, the transgenic mammals provide the ability to visualize cells with lower promoter activation levels, which would otherwise be overwhelmed in prior art methods using in situ hybridization, or with immunohistochemistry using an antibody against a protein epitope. For instance, GLT-1 has an extremely high level of expression in brain tissue (approx 1% of total protein) and exhibits a strong staining phenotype using standard immunohistochemical techniques. Therefore, it is difficult to differentiate individual cellular patterns of expression where the transporter could also be playing an important role.

The invention also includes the identification of distinct cells types exhibiting distinct patterns of GLAST and/or GLT-1 promoter activity. That is, the invention relates to the observation using the transgenic mammals of the invention that distinct cell types exhibit both overlapping and non-overlapping activation of GLAST and/or GLT-1 promoters. The disclosure presented herein demonstrate that both GLAST and GLT-1 promoters are active in glial fibrillary acidic protein (GFAP) positive astrocytes of the hippocampus dentate gyrus, as well as in the radial glia, but there are cells in the granule cell layer in which the GLAST promoter was observably active independent of the GLT-1 promoter or GFAP protein (marker for astrocytes). There is therefore a population of cells in the granule cell layer that are non-astrocytic GLAST expressing cells. As such, the invention includes a population of GLAST promoter positive cells that are negative for GLT1 promoter expression and NeuN (a neuronal marker) present in the granule cell layer.

The invention also includes a population of cells present in the cortex where the GLT-1 promoter is not activated but the GLAST promoter is activated. These cells are small round cells that resemble neurons or neuron progenitors based on co-staining with the NeuN antibody (a neuronal marker). A large number of GLAST-promoter promoter active cells in the cortex also overlapped with oligodendrocytes (PLP promoter reporter positive) depending on developmental stage. Since a number of the GLAST positive cell were not astrocytes or neurons, the present invention includes a population of oligodendroglias, wherein GLAST promoter was activated and GLT-1 promoter was not activated. In fact, in the striatum, many GLAST promoter active cells overlapped with the PLP oligodendrocyte promoter reporter during later stages of development, although this expression was not observed in young mammals.

In the spinal cord, the expression of GLT-1 and GLAST promoter reporters was completely separate, with the vast majority of gray matter astrocytes expressing GLT-1 promoter. The GLAST expressing cells were pronounced in the white matter tracts, and many of these cells appeared to be oligodendroglia, but not neurons or microglia.

In multiple brain regions, GLAST+/GLT1-cells were often observed to be associated together, adjacent to neuronal somas. These cells were not oligodroglia (PLP-) and did not stain for GFAP or microglial markers.

GLAST promoter activity, unlike GLT1 promoter activity, appears to be expressed by progenitor cells in the early postnatal period. It was observed that GLAST positive cells were intensely localized to the periventricular white and gray matter in early postnatal time points. Even at later postnatal time points, GLAST was expressed by NeuN positive cells periventricularly, although fully disappeared in adult brain.

The invention includes a population of cells having distinct expression patterns of GLT-1 and/or GLAST promoter activity. The invention also provides a method of obtaining such populations of cells from a sample of cells obtained from the transgenic mammal. Such method comprises isolating a population of cells from the mammal, assaying for expression of the reporter marker in the cells, wherein the cells which express the detectable marker can then be isolated from the population of cells to provide a sub-population of cells. The cells can also be isolated on the basis of distinct cell type markers such as GFAP (astrocytes), PLP (oligodendrocytes) and NeuN (neurons). Further, the cells of the invention can be isolated from the transgenic mammal using any combination of the reporter marker and/or cell type markers.

The invention also includes a method for producing a transgenic mouse cell line that expresses a reporter gene operatively linked to a glutamate transporter promoter, said method comprising: (a) isolating cells from the transgenic mouse of the invention; and (b) placing the isolated cells under conditions to maintain growth and viability of the isolated cells such that said transgenic mouse cell line expresses said reporter nucleic acid.

Cells of the CNS can be obtained from the central nervous system of a mammal from a variety of tissues including but not limited to, fore brain, hind brain, whole brain and spinal cord. The cells can be isolated and cultured using the methods detailed elsewhere herein or using methods known in the art, for example using methods disclosed in U.S. Pat. No. 5,958,767 hereby incorporated herein in its entirety by reference. In one example, tissue from brain is removed using sterile procedures, and the cells are dissociated using any method known in the art including treatment with enzymes such as trypsin, collagenase and the like, or by using physical methods of dissociation such as mincing or treatment with a blunt instrument. Dissociation of CNS derived cells, and other multipotent stem cells, can be carried out in a sterile tissue culture medium. Dissociated cells are centrifuged at low speed, between 200 and 2000 rpm, usually between 400 and 800 rpm, the suspension medium is aspirated, and the cells are then resuspended in culture medium.

Cells that express the reporter marker (i.e. fluorescent protein) can be isolated using fluorescence activated cell sorter as described by Roy et al. (2000, J Neurosci Res. 59:321-331). Examples of such fluorescent protein include, but are not limited to luciferase protein and green fluorescent protein (Heim et al., 1996, Curr. Biol. 6:178-182).

According to the present invention, the phenotype of a cell, including but not is limited to the expression of the reporter gene and cell surface markers, can be used as unique markers for identifying and isolating a desired cell type. That is, the unique cell surface markers in combination with the expression of reporter genes (or otherwise activity of GLT-1 and/or GLAST promoter) on the cells of the present invention can be used to isolate a specific population of cells from a mixed population of cells derived from the transgenic mammal. One skilled in the art would appreciate that an antibody specific for a cell surface marker can be conjugated to a physical support (i.e. a streptavidin bead) and therefore provide the opportunity to isolate cell surface specific cells. The isolation of cells based on surface markers can be combined with the isolation of cells based on the reporter marker (i.e. fluorescent protein). For example, the cells can be isolated using a flow cytometry-based cell sorter. The isolated cell can then be cultured and expanded in vitro using methods disclosed herein or conventional methods.

A further embodiment of the present invention encompasses a method of depleting or separating a population of cells derived from the transgenic mammal. A specific cell population can be depleted from a mixed cell population by incubating an antibody that specifically binds to a desired cell type (i.e. astrocytes, oligodendrocytes, neurons or progenitor cells) within the mixed populations followed by a separation step including but not limited to magnetic separation. The process of magnetic separation is accomplished by using magnetic beads, including but not limited to Dynabeads® (Dynal Biotech, Brown Deer, Wis.). MACS separation reagents (Miltenyi Biotec, Auburn, Calif.) can also be used to deplete a desired cell population from a mixed population of cells. Following the separation of the desired cell type, the cells can be cultured in vitro to enrich the cell population for experiemental/therapeutic use. As such, the invention includes an enriched population of a desired cell type.

In yet another embodiment, the cells isolated are glial-restricted precursor cells. Such cells retain the properties of an undifferentiated glial-restricted precursor cell and can undergo differentiation into oligodendrocytes and in some instances into astrocytes. These cells are multipotent having the ability of differentiating into more than one cell type. Preferably, the undifferentiated glial-restricted precursor cell is isolated from the transgenic mammal of the invention including but not limited to the GLAST BAC promoter reporter mouse, the GLT BAC promoter reporter mouse, and the double crossed GLAST/GLT BAC promoter reporter mouse.

The isolated cells of the invention can also be immortalized. Because primary cells can reach senescence after a limited number of population doublings, it is advantageous to establish an immortalized cell line having an extended replicative capacity. The use of an immortalized cell line provides a consistent material throughout a research project.

Some cells immortalize spontaneously by passing through replicative senescence and thus easily adapt to conditions in culture. However, these spontaneously immortalized cells invariably have unstable genotypes and are host to numerous genetic mutations, rendering them less reliable representatives of their starting tissue's phenotype. The ideal immortalization protocol, therefore, would produce cells that are not only capable of extended proliferation, but also possess the same genotype and tissue markers of their parental tissue.

Several methods exist for immortalizing mammalian cells in culture. Viral genes, including v-myc, Epstein-Barr virus (EBV), Simian virus 40 (SV40) T antigen, adenovirus E1A and E1B, and human papillomavirus (HPV) E6 and E7, can induce immortalization by a process known as “viral transformation.” For the most part, these viral genes achieve immortalization by inactivating the tumor suppressor genes that put cells into a replicative senescent state.

Another method of generating an immortalized cell line is through expression of the telomerase reverse transcriptase protein (TERT), particularly those cells most affected by telomere length. This protein is inactive in most somatic cells, but when hTERT is exogenously expressed, the cells are able to maintain telomere lengths sufficient to avoid replicative senescence. Analysis of several telomerase-immortalized cell lines has verified that the cells maintain a stable genotype and retain critical phenotypic markers.

Whether the cells are primary cells or immortalized, any medium-capable of supporting cells derived from the CNS tissue in cell culture may be used to culture the cells. Media formulations that support the growth of cells derived from the CNS include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BMB—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non-essential amino acids), and the like.

Additional non-limiting examples of media useful in the methods of the invention can contain a source of growth factors such as fetal serum of bovine or other species at a concentration at least 1% to about 30%, preferably at least about 5% to 15%, most preferably about 10%. Embryonic extract of chicken or other species can be present at a concentration of about 1% to 30%, preferably at least about 5% to 15%, most preferably about 10%.

In another aspect, the medium comprises a standard culture medium that is serum-free (containing 0-0.49% serum) or serum-depleted (containing 0.5-5.0% serum), known as a basal medium, such as Iscove's modified Dulbecco's medium (“IMDM”), RPMI, DMEM, DMEM/F12, Fischer's, alpha medium, Leibovitz's, L-15, NCTC, F-10, F-12, MEM and McCoy's; a suitable carbohydrate source, such as glucose; a buffer such as MOPS, HEPES or Tris, preferably HEPES; and one or more growth factors that stimulate proliferation of neural stem cells, such as EGF, bFGF, platelet derived growth factor (PDGF), nerve growth factor (NGF), and analogs, derivatives and/or combinations thereof, preferably EGF and bFGF in combination Standard culture media typically contains a variety of essential components required for cell viability, including inorganic salts, carbohydrates, hormones, essential amino acids, vitamins, and the like. Preferably, DMEM or F-12 is the standard culture medium, most preferably a 50/50 mixture of DMEM and F-12. Both media are commercially available (DMEM; GIBCO, Grand Island, N.Y.; F-12, GIBCO, Grand Island, N.Y.). A premixed formulation of DMEM/F-12 is also available commercially. It is advantageous to provide additional glutamine to the medium. It is also advantageous to provide heparin in the medium. It is further advantageous to add sodium bicarbonate to the medium. It is also advantageous to add N2 supplement. Preferably, the conditions for culturing the cells should be as close to physiological conditions as possible.

Following isolation, the cells are incubated in a cell medium in a culture apparatus for a period of time or until the cells reach confluency before passing the cells to another culture apparatus. Following the initial plating, the cells can be maintained in culture for a period of about 6 days to yield the Passage 0 (P0) population. The cells can be passaged for an indefinite number of times, each passage comprising culturing the cells for about 6-7 days, during which the cell doubling times can range between 3-5 days. The culturing apparatus can be of any culture apparatus commonly used in culturing cells in vitro.

The cells can be cultured in standard cell medium supplemented with N2 supplement until the cells reach confluency. Preferably, the level of confluence is greater than 70%. More preferably, the level of confluence is greater than 90%. A period of time can be any time suitable for the culture of cells in vitro. The cell medium may be replaced during the culture of the cells at any time. Preferably, the cell medium is replaced every 3 to 4 days. The cells are then harvested from the culture apparatus whereupon the cells can be used immediately or cryopreserved to be stored for use at a later time. The cells may be harvested by trypsinization, EDTA treatment, or any other procedure used to harvest cells from a culture apparatus.

The cells described herein may be cryopreserved according to routine procedures. Preferably, about one to ten million cells are cryopreserved in cell medium containing 10% DMSO in vapor phase of Liquid N₂. Frozen cells can be thawed by swirling in a 37° C. bath, resuspended in fresh growth medium, and grown as usual.

Screening Assays

The invention provides a method (also referred to herein as a “screening assay”) for identifying modulators, i.e., candidate or test compounds or agents (e.g., nucleic acids, peptides, peptidomimetics, small molecules, or other drugs) which bind to the GLT-1 and/or GLAST promoter and can have a stimulatory or inhibitory effect on, for example, GLT-1 and/or GLAST promoter activity.

In one embodiment, the invention provides assays for screening candidate or test compounds which are capable of modulating GLT-1 and/or GLAST promoter activity. In another embodiment, the invention provides assays for screening candidate or test compounds which bind to or otherwise modulate the activity of a GLT-1 and/or GLAST promoter. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Nati. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In a preferred embodiment, an assay is a cell-based assay in which a cell (e.g., a cell isolated from a BAC-reporter transgenic mammal) which expresses a reporter gene operatively linked to a GLT-1 or GLAST promoter or portion thereof (e.g., whose expression is under the control of the GLT-1 or GLAST promoter or portion thereof) is contacted with a test compound and the ability of the test compound to modulate GLT-1 or GLAST promoter activity is determined. Determining the ability of the test compound to modulate GLT-1 or GLAST promoter activity can be accomplished by monitoring reporter gene expression (e.g., reporter mRNA or polypeptide expression level) or activity. As described elsewhere herein, the reporter can be any detectable marker. For example, the reporter can be a nucleic acid sequence, the expression of which can be measured by, for example, Northern blotting, RT-PCR, primer extension, or nuclease protection assays. The reporter may also be a nucleic acid sequence that encodes a polypeptide, the expression of which can be measured by, for example, Western blotting, ELISA, or RIA assays. Reporter expression can also be monitored by measuring the activity of the polypeptide encoded by the reporter using, for example, a standard glutamate transport assay, a luciferase assay, a β-galactosidase assay, a chloramphenicol acetyl transferase (CAT) assay, or a fluorescent protein assay. The cell may be any cell that expresses a reporter under the control of the GLT-1 or GLAST promoter.

A BAC-reporter transgenic mammal (i.e. GLT-1 BAC-reporter transgenic mammal) can be crossed with a different BAC-reporter transgenic mammal (i.e. GLAST BAC-reporter transgenic mammal) to obtain a double BAC-reporter transgenic mammal. Accordingly, a cell exhibiting both GLT-1- and GLAST-reporter activity can be isolated from such a double BAC-reporter transgenic mammal and used in the screening assays discussed herein. That is, it is possible to compare expression of both promoters in specific cells.

The level of expression or activity of a reporter under the control of the GLT-1 or GLAST promoter in the presence of the candidate compound is compared to the level of expression or activity of the reporter in the absence of the candidate compound. The candidate compound can then be identified as a modulator of GLT-1 and/or GLAST promoter activity based on this comparison. For example, when expression of reporter mRNA or protein expression or activity is greater (statistically significantly greater) in the presence of the candidate compound than in its absence, the candidate compound is identified as a stimulator of GLT-1 and/or GLAST promoter activity. Alternatively, when expression or activity of reporter mRNA or protein is less (statistically significantly less) in the presence of the candidate compound than in its absence, the candidate compound is identified as an inhibitor of GLT-1 and/or GLAST promoter activity.

The ability of the test compound to bind to the promoter and/or to modulate the binding of proteins (e.g., transcription factors) to the promoter can also be determined. Determining the ability of the test compound to bind to and/or modulate GLT-1 and/or GLAST promoter binding to a binding protein can be accomplished, for example, by coupling the test compound, the GLT-1 and/or GLAST promoter, or the binding protein with a radioisotope/enzymatic label such that binding of the GLT-1 and/or GLAST promoter to the test compound or the binding protein can be determined by detecting the labeled component in a complex. For example, compounds (e.g., the test compound, the appropriate glutamate transporter promoter, or a binding protein) can be labeled with ³²P, ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of the present invention to determine the ability of a compound (e.g., a test compound or GLT-1 and/or GLAST promoter binding protein) to interact with the GLT-1 and/or GLAST promoter without the labeling of any of the interactants. For example, a microphysiometer can be used to detect the interaction of a compound with the GLT-1 and/or GLAST promoter without the labeling of either the compound or the GLT-1 and/or GLAST promoter (McConnell et al., 1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and the GLT-1 and/or GLAST promoter.

In yet another aspect of the invention, the BAC-reporter transgenic cells can be used as “bait” in a one-hybrid assay (see, e.g., BD Matchmaker One-Hybrid System (1995) Clontechniques X(3):2-4; BD Matchmaker Library Construction & Screening Kit (2000) Clontechniques XV(4):5-7; BD SMART technology overview (2002) Clontechniques XVII(1):22-28; Ausubel, F. M., et al. (1998 et seq.) Current Protocols in Molecular Biology Eds. Ausubel, F. M., et al., pp. 13.4.1-13.4.10) to identify proteins which bind to or interact with the GLT-1 and/or GLAST promoter. Such proteins are considered to be GLT-1 and/or GLAST promoter-binding proteins or GLT-1 and/or GLAST promoter-bp. These binding proteins are involved in GLT-1 and/or GLAST promoter activity. Such GLT-1 and/or GLAST promoter-binding proteins are also likely to be involved in the regulation of transcription from the GLT-1 and/or GLAST promoters.

In another aspect, the invention pertains to a combination of two or more of the assays described herein. For example, a modulating agent can be identified using a cell-based or a cell-free assay, and the ability of the agent to modulate the activity of a GLT-1 and/or GLAST promoter can be confirmed in vivo, e.g., in an mammal such as an mammal model for a neurological disease.

This invention further pertains to novel agents identified by the above-described screening assays. Accordingly, it is within the scope of this invention to further use an agent identified as described herein in an appropriate mammal model (e.g., an mammal model for a neurological disease). For example, an agent identified as described herein (e.g., a GLT-1 and/or GLAST promoter modulating agent or a GLT-1 and/or GLAST promoter binding protein) can be used in an mammal model to determine the efficacy, toxicity, or side effects of treatment with such an agent. Alternatively, an agent identified as described herein can be used in an mammal model to determine the mechanism of action of such an agent. Furthermore, this invention pertains to uses of novel agents identified by the above-described screening assays for treatments as described herein.

Methods of Treatment

In addition to screening compounds for the ability to modulate a glutamate promoter, the invention also encompasses a method of identifying a compound useful for treating a neurological and/or psychiatric disorder. Assessing the expression and/or activity of a glutamate transporter promoter (i.e. GLT-1 and/or GLAST promoter) can be performed by assessing, among other things, the levels of the reporter marker or the mRNA that encodes it in a cell or tissue, and the like, and then the level can be compared to the level in an otherwise identical cell or tissue to which the compound is not administered. Alternatively, the level of the reporter marker or the mRNA that encodes it in a cell or tissue contacted with a compound can be compared with the level of the reporter marker or the mRNA that encodes it in the cell or tissue prior to administration of the compound. Such a compound can be a useful therapeutic for treating and/or preventing a neurological and/or psychiatric disorder. In some instances, the disorder is associated with a defect in the expression of a glutamate transporter. In another instance, the disorder is associated with a defect in the biological activity of the glutamate transporter.

Additionally, a cell or tissue comprising the BAC promoter sequence operatively linked to a reporter gene sequence of the invention can be contacted with a compound, and the level of reporter molecule can be assessed and compared to the level of the otherwise identical reporter molecule in the cell and/or tissue prior to administration of the compound. Further, the level of the reporter molecule can be compared to the level of the reporter molecule in an otherwise identical cell or tissue not contacted with the compound.

The invention includes a method of identifying a compound useful for treating a neurological and/or psychiatric disorder in a mammal. The method comprises identifying a compound that modulates the activity of GLT-1 and/or GLAST promoter in a mammal (including in a cell or tissue thereof), preferably in the CNS. The ability of a compound to modulate the activity of GLT-1 and/or GLAST promoter provides a therapeutic benefit thereby treating or preventing a neurological and/or psychiatric disorder mediated by or associated with abnormal GLT-1 and/or GLAST promoter activity. This is because abnormal level of GLT-1 and/or GLAST promoter activity is associated with, or mediates, such disorder and that modulating GLT-1 and/or GLAST promoter activity prevents and/or treats the disorder.

A compound that modulates GLT-1 and/or GLAST promoter activity is a powerful therapeutic or prophylactic treatment of a neurological and/or psychiatric disorder, such that identification of such a compound identifies a potential therapeutic for such disease.

Following the identification of a compound using the transgenic mammal and cells isolated therefrom, the compound can be administered to another mammal (e.g., a human) in order to assess whether the identified compound can modulate the level of GLT-1 and/or GLAST promoter activity in the mammal. One way of assessing the activity of the compound is to compare the level of GLT-1 and/or GLAST promoter activity in the mammal before and after administration of the compound. A higher level of the appropriate glutamate transporter protein or the mRNA that encodes the corresponding glutamate transporter protein in the mammal after administration of the compound compared with the level of the glutamate transporter protein or the corresponding mRNA before administration of the compound indicates that the compound is useful for treating a neurological and/or psychiatric disorder associated with decreased activity of a glutamate transporter promoter in a mammal.

A lower level of the appropriate glutamate transporter protein or the corresponding mRNA that encodes the glutamate transporter protein in the mammal after administration of the compound compared with the level of the glutamate transporter protein or the corresponding mRNA before administration of the compound indicates that the compound is useful for treating a neurological and/or psychiatric disorder in a mammal associated with increased activity of a glutamate transporter promoter.

In one embodiment, the present invention provides methods of treating neurological and/or psychiatric disorders which comprise administering a therapeutically effective amount of a pharmaceutical composition comprising an GLT-1 and/or GLAST promoter modulator to a subject (e.g., a mammal such as a human).

To modulate GLT-1 and/or GLAST promoter activity, and thereby modulate GLT-1 and/or GLAST gene expression, a compound disclosed herein or identified by the screening assays of the invention, can be administered to a cell or a subject. Administration of a GLT-1 and/or GLAST promoter modulator to mammalian cells (including human cells) can modulate (e.g., up- or down-regulate) GLT-1 and/or GLAST mRNA and/or polypeptide expression, thereby up- or down-regulating glutamate transport into the cell. In such methods, the GLT-1 and/or GLAST promoter modulator can be administered to a mammal (including a human) by known procedures.

The preferred therapeutic methods of the invention (which include prophylactic treatment) in general comprise administration of a therapeutically effective amount of a GLT-1 or GLAST promoter modulator to an mammal in need thereof, including a mammal, particularly a human. Such treatment will be suitably administered to subjects, particularly humans, suffering from, having, susceptible to, or at risk for a neurological and/or psychiatric disorder. The GLT-1 and/or GLAST promoter modulator of the invention may be also used in the treatment of any other disorders in which GLT-1 and/or GLAST may be implicated.

The compounds of this invention may be modified by appending appropriate functionalities to enhance selective biological properties. Such modifications are known in the art and include those which increase biological penetration into a given biological compartment (e.g., central nervous system), increase oral availability, increase solubility to allow administration by injection, alter metabolism and alter rate of excretion.

It will be appreciated that actual preferred amounts of a given GLT-1 and/or GLAST modulator of the invention used in a given therapy will vary according to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests, or by any method known in the art or disclosed herein.

For example, in certain embodiments, of this invention, a level of GLT-1 and/or GLAST expression or activity in a subject is determined at least once. Comparison of GLT-1 and/or GLAST levels, e.g., to another measurement of GLT-1 and/or GLAST level obtained previously or subsequently from the same patient, another patient, or a normal subject, may be useful in determining whether therapy according to the invention is having the desired effect, and thereby permitting adjustment of dosage levels as appropriate. Determination of GLT-1 and/or GLAST expression levels may be performed using any suitable sampling/expression assay method known in the art or otherwise described herein. Preferably, a tissue or fluid sample is first removed from a subject. Examples of suitable samples include blood, mouth or cheek cells, and hair samples containing roots. Other suitable samples would be known to the person skilled in the art. Determination of protein levels and/or mRNA levels (e.g., GLT-1 and/or GLAST levels) in the sample can be performed using any suitable technique known in the art, including, but not limited to, ELISA, blotting/chemiluminescence methods, real-time PCR, and the like.

Certain compounds of the invention can conventionally be administered for the treatment or prevention of a neurological and/or psychiatric disorder. In certain embodiments of the present invention, the compounds are administered for prophylaxis or treatment of diseases or disorders associated with neurodegeneration.

For therapeutic applications, GLT-1 and/or GLAST modulators of the invention may be suitably administered to a subject such as a mammal, particularly a human, alone or as part of a pharmaceutical composition, comprising the GLT-1 and/or GLAST modulator together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics often will be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

It will be appreciated that actual preferred amounts of a given GLT-1 and/or GLAST modulator of the invention used in a given therapy will vary to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests.

The present invention also includes methods for monitoring the effects of agents (e.g., drugs) on the activity of the GLT-1 and/or GLAST promoters. Monitoring the influence of agents on the activity of the GLT-1 and/or GLAST promoters can be applied not only in basic drug screening, but also in clinical trials. For example, the effectiveness of an agent determined by a screening assay as described herein to increase GLT-1 or GLAST promoter activity, can be monitored in clinical trials of subjects exhibiting decreased GLT-1 or GLAST promoter activity. Alternatively, the effectiveness of an agent determined by a screening assay to decrease GLT-1 or GLAST promoter activity, can be monitored in clinical trials of subjects exhibiting increased GLT-1 or GLAST promoter activity. In such clinical trials, the expression or activity of an GLT-1 or GLAST promoter, and preferably, other genes that have been implicated in, for example, a neurological and/or psychiatric disorder can be used as a “read out” or markers of the phenotype of a particular cell.

For example, and not by way of limitation, genes, including GLT-1 and/or GLAST, that are modulated in cells by treatment with an agent (e.g., compound, drug or small molecule) which modulates GLT-1 and/or GLAST promoter activity (e.g., identified in a screening assay as described herein) can be identified. Thus, to study the effect of agents on neurological and/or psychiatric disorders, for example, in a clinical trial, cells can be isolated and RNA prepared and analyzed for the levels of expression of GLT-1 and/or GLAST and other genes implicated in the disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of GLT-1 and/or GLAST or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of GLT-1 and/or GLAST protein, mRNA, and/or genomic DNA in the preadministration sample; (iii) obtaining one or more post-administration samples from the subject; (iv) detecting the level of expression or activity of the GLT-1 or GLAST protein, mRNA, and/or genomic DNA in the post-administration samples; (v) comparing the level of expression or activity of the GLT-1 and/or GLAST protein, mRNA, and/or genomic DNA in the pre-administration sample with the GLT-1 and/or GLAST protein, mRNA, and/or genomic DNA in the post administration sample or samples; and (vi) altering the administration of the agent to the subject accordingly. For example, increased administration of the agent may be desirable to increase the expression or activity of GLT-1 and/or GLAST to higher levels than detected, i.e., to increase the effectiveness of the agent. Alternatively, decreased administration of the agent may be desirable to decrease expression or activity of GLT-1 and/or GLAST to lower levels than detected, i.e., to decrease the effectiveness of the agent. According to such an embodiment, GLT-1 and/or GLAST expression or activity may be used as an indicator of the effectiveness of an agent, even in the absence of an observable phenotypic response.

Therapeutic Use

By way of example, Amyotrophic lateral sclerosis (ALS) which is the most common form of adult motor neuron disease in which there is progressive degeneration of both the upper motor neurons in the cortex and the lower motor neurons in the brain stem and spinal cord. The present invention provides a therapeutic use of the compounds identified by the screening methods disclosed herein. However, the invention should not be limited to only treating ALS, but rather any neurological and/or psychiatric disorder.

The majority of ALS cases (95%) are apparently sporadic (SALS), while approximately 5% are familial (FALS). FALS cases were found to be associated with mutations in SOD-1, the gene that encodes copper-zinc superoxide dismutase (CuZnSOD). Common to both familial and sporadic ALS is the loss of the astroglial glutamate transporter GLT-1/EAAT2 protein. As discussed elsewhere herein, GLT-1/EAAT2 protein is the predominant protein responsible for the bulk of synaptic clearance of glutamate. In particular, GLT-1/EAAT2 protein protects against excitotoxic neurodegeneration. Measurement of functional glutamate transport in ALS tissue revealed a marked diminution in the affected ALS brain regions. The loss of functional glutamate transporter is likely the result of a dramatic loss of astroglial glutamate transporter protein GLT-1/EAAT2 protein, which can be in up to 65% of sporadic ALS patients. Regardless of the mechanism, lowering GLT-1/EAAT2 protein with antisense oligonucleotides has demonstrated that loss of transport activity directly provokes neuronal death. These and other studies suggest that the functional loss of GLT-1/EAAT2 protein (associated with astrocyte dysfunction), contributes to the loss of motor neurons in both inherited and sporadic ALS.

The invention includes compounds identified using the transgenic mammal and/or cells isolated therefrom to screen for candidate compounds having the ability to increase the activity of the GLT-1 promoter. Such a compound can be used as a therapeutic to treat a neurological disorder such as ALS, or otherwise a disorder associate with decreased activity of GLT-1 promoter. That is, this glutamate transporter has been demonstrated to be important for normal excitatory synaptic transmission, while its dysfunction is implicated in a neurological disorder including, but not limited to ALS, multiple sclerosis, Alzheimer's, stroke, brain tumors and epilepsy. Therefore, the ability for a compound to increase the activity of GLT-1 promoter and/or the function of the glutamate transporter can provide a therapeutic to protect or otherwise prevent glutamate neurotoxicity with respect to decreased GLT-1 promoter activity and/or dysfunction of the glutamate transporter protein. Of course the invention should not be limited to diseases and disorders associated with GLT-1, but should be associated with diseases and disorders associated with GLT-1 and/or GLAST.

The invention also includes compounds identified using the screening methods disclosed herein. In some aspects, the invention includes compounds having the ability to decrease the activity of the glutamate transporter promoter. Such a compound can be used as a therapeutic to treat a neurological and/or psychiatric disorder associated with increased activity of the glutamate transporter promoter and/or increase amount of the glutamate transporter. Without wishing to be bound by any particular theory, such a disease or disorder can arise from the administration of a compound having the ability to increase the desired glutamate transporter promoter. Therefore, the invention encompasses compounds having the ability of decreasing the same glutamate transporter promoter having previously increased with the compound. Therefore, the ability for a compound to decrease the activity of GLT-1 and/or GLAST promoter and/or the function of the respective glutamate transporter can provide a therapeutic against a disorder associated with excessive promoter activity.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Generation of BAC Transgenic Mice for GLAST and GLT-1

The BAC transgenic mice are useful for studying the biology of glutamate transport and generating cells for screening compounds capable of activating the GLAST and GLT-1 genes. The transgenic mice were generated carrying bacterial artificial chromosomes (BACs) containing the mouse GLAST or GLT-1 genes plus at least 40 kB of DNA upstream of the first exon, all of the introns, and at least 20 kb of DNA downstream of the final exon. In each case, eGFP cDNA has been placed within the initiating ATG codon so that when the promoter is activated, fluorescent protein is produced. Mouse BACs RPCI-24-287G1 1 and RPCI-23-361H22, both obtained from BAC-PAC Resources (Oakland, Calif.; clones may also be referred to as RP24-287G11 and RP23-361H22, respectively), were used for the GLAST and GLT-1 reporter mice, respectively. The GLAST BAC sequence is on mouse chromosome 15; the GLAST is gene slcla3 on mouse chromosome 15 at locus position 8425087-8425252 in Build 34.1. The GLT-1 BAC sequence is on mouse chromosome 2; the GLT-1 is gene slcla2 on mouse chromosome 2 at locus position 102411193-102482861 in Build 34.1 (See Waterston et al., 2002, Nature 420:520-62).

The RP24-287G1 1 (GLAST) clone was derived from the RPCI-24 BAC library, which was constructed from tissues derived from single male C57BL/6J mouse. Spleen and brain genomic DNA samples were isolated and partially digested with MboI. Size-selected MboI fragments were cloned into the pTARBAC1 vector between the BamHI sites. The ligation products were transformed into DH10B electrocompetent cells (BRL Life Technologies).

The RPCI-23-361H22 (GLT-1) clone was derived from the RPCI-23 BAC library, which was constructed at the Roswell Park Cancer Institute from pooled tissues derived from three female C57BL/6J mice. Mouse kidney and brain genomic DNA samples were isolated and partially digested with a combination of EcoRI and EcoRI Methylase. Size-selected EcoRI fragments were cloned between the pBACe3.6 vector at the EcoRI sites. The ligation products were transformed into DH1OB electrocompetent cells (BRL Life Technologies).

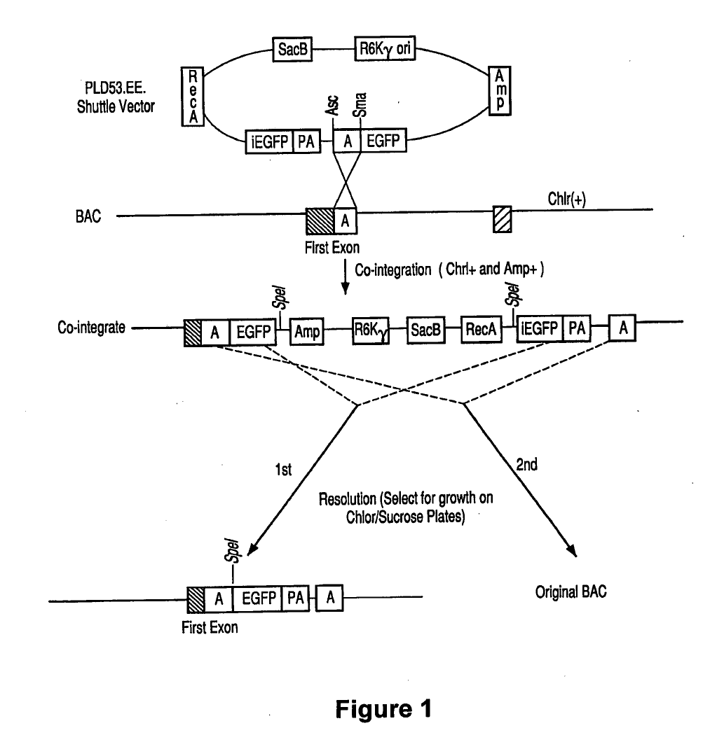

The procedure for BAC modification was adapted from the procedure described by Gong, S. et al. (Gong et al., 2002, Genome Res. 12:1992-1998, incorporated herein by reference), with minor alterations (see FIG. 1). First, a ˜500 bp gene-specific region (the “A box”), including a disruption of the initiating ATG, was inserted into the shuttle vector pLD53SC.E-E, which contains two copies of reporter eGFP cDNA. This shuttle vector was then integrated into the BAC via homologous recombination. Successful co-integrates were selected, and a second recombination event between the two eGFP regions was performed to eliminate extraneous vector sequences. The final modified BAC was linearized and injected into mouse pronuclei for generation of transgenic reporter mice. Founders were identified via PCR to detect eGFP transcripts. These founders were bred with wild type mice to generate independent F1 lines.

After determining, via Southern blots and eGFP fluorescence levels, which lines demonstrated the highest reporter level, primary astrocyte cultures for ongoing drug studies were prepared following standard procedures. Early post-natal day mice (usually post-natal day 3, but up to post-natal day 12) were anesthetized and decapitated. In a laminar flow hood, brains were removed from the skulls and placed in a Petri dish containing calcium/magnesium free Hanks balanced salt solution (HBSS). The cerebellum, brain stem, and other brain tissue other than the cerebral cortex, then the meninges, were removed, and the tissue was cut into small pieces. Dissociation was accomplished by incubation in trypsin/DNAse I, followed by trituration, washing, and filtration. The dissociated cells were suspended in MEM plus 10% horse serum, 1% pen/strep/glutamine, and 1% pyruvate and plated.

A. Material and Methods

BAC Construct

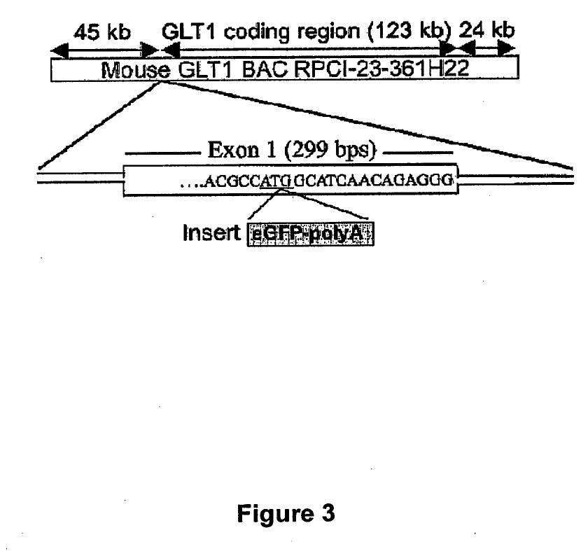

Mouse BACs RPCI-24-287G11 and RPCI-23-361H22, both obtained from BAC-PAC Resources, were used for the GLAST and GLT-1 reporter mice, respectively. BAC RPCI-24-287G11, containing the entire GLAST gene plus 18 kb upstream of the first exon and 60 kb downstream of the last exon, was modified using a double homologous combination approach with the SV-RecA shuttle vector as described by Yang et al. (1997, Nat. Biotechnol. 1:859-865, incorporated herein by reference), to insert cDNA for DsRed. The first homology region, termed the A-box, included the 721 base pairs between the sequence 5′-TTCCCTGTAAAAGCCTCAATT-3′ (SEQ ID NO:1) and the reverse complement sequence 5′-GCTCTTCTCCGTTGCTTTTGGTCAT-3′ (SEQ ID NO:2) based on the published online mouse genome (http://www.ensembl.org/Mus_musculus/index.html. The second homology region, the B-box, included the 618 base pairs between the sequence 5′-AAGACCAAAAGCAACGGAG-3′ (SEQ ID NO:3) and the reverse complement sequence 5′-TGCTGGGGTTAAAGGTGTG-3′ (SEQ ID NO:4). The GLAST start codon, in exon 2, was mutated to AAG, and the DsRed cDNA (DsRed Express, Clontech) was inserted downstream, immediately after the end of the A-box, within the same exon, as illustrated in FIG. 3.

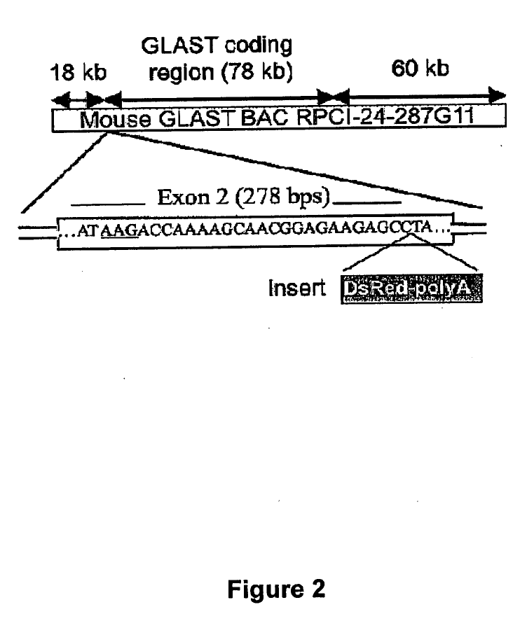

BAC RPCI-23-361H22, containing the entire 123-kb GLT-1 gene plus 45 kb upstream of the first exon and 24 kb downstream of the last exon, was modified using the pLD53SC.E-E shuttle vector as described by Gong et al. (2002, Genome Res. 12:1992-1998, incorporated herein by reference) with minor alterations, to insert cDNA for eGFP between the AT and the G of the start codon (FIG. 2). The A-box homology region was cloned into the pLD53SC.E-E shuttle vector using the AscI and SmaI sites on the vector. The 502-base pair A-box was generated with PCR primers 5′-AGGCGCGCCCAGGGCGCAGCGGCCTCT-3′ (SEQ ID NO:5) (AscI site underlined) and 5′-TTCCCCCGGGATGGCGTGGGGAACGCCC-3′ (SEQ ID NO:6) (SmaI site underlined) based on the sequence upstream of the GLT-1 Exon 1 initiation codon found in the published online mouse genome (http://www.ensembl.org/Mus_musculus/index.html.

The final modified BACs were transfected into HEK-293 cells and primary astrocyte cultures using Fugene 6 Transfection Reagent (Roche) to validate reporter function, linearized, and injected into mouse pronuclei for generation of transgenic reporter mice. Founders were identified via PCR to detect DsRed (GLAST) or eGFP (GLT-1) transcripts. These founders, of the B6SJL hybrid strain, were bred with C57B1/6 wild type mice to generate independent F1 lines.

Western Blotting

Mice were anesthetized and sacrificed, and either brain cortex (for GLT-1) or cerebellum (for GLAST) was dissected out and homogenized in standard lysis buffer. After sonication, protein concentration was determined by performing a Bradford Assay (Pierce) and then diluted 1:1 in sample buffer (250 mM Tris-HCl, pH 6.8, 4% SDS, 10% glycerol, and 1% β-mercaptoethanol). Following denaturation by heating at 95° C. for 3 minutes, 10 μg of protein were loaded in each lane of a 4-10% polyacrylamide gel (Invitrogen) along with a protein standard ladder. Non-specific sites were blocked with 5% non-fat milk, and blots were incubated overnight at 4° C. with rabbit polyclonal anti-actin to control for protein loading levels, and either anti-GLAST (concentration (1:100), epitope) or anti-GLT-1 (concentration (1:25,000), epitope) antibodies. Antibody labeling was detected by incubating blots with horseradish peroxidase-labeled anti-rabbit secondary antibody and visualized with Enhanced Chemiluminescence (Amersham Pharmacia).

Histology and Quantification

Mice were anesthetized and perfused with 4% paraformaldehyde via intra-ventricular cannulation, post-fixed in the same fixative overnight, cryoprotected with 20% glycerol in phosphate buffer for 24 hours, and then frozen. Sections were cut at 40-45 μm, and mounted on a slide. Digital images were taken from either a Nikon E800 microscope, or a Zeiss LSM 510 Meta confocal microscope. Relative activation levels of the GLT-1 promoter were quantified using Image J software (NIH).

Immunohistochemistry

Sections were incubated overnight in either GFAP (Sigma) or Neu N (Chemicon) antibodies. After several rinses the sections were incubated with secondary antibody conjugated to fluorescent label AMCA (blue). GFAP (Sigma, 1:500), NeuN (Chemicon, 1:2000), Ibal (WAKO, 1 μg/ml), Nestin (Chemicon, 1: 100), NG2 (1:2000), secondary (AMCA conjugated, 1:200, Vector Labs).

Electrophysiology

To check for functional expression in non-astrocytic cells with GLAST promoter activity, and to compare currents in these cells to that in astrocytes, whole cell voltage clamp recordings from acute slices were conducted. The results were examined for their response to laser-pulse photolysis of caged D-aspartate (Huang et al., 2005, Biochemistry 44:3316-3326); D-aspartate is a high affinity substrate of glutamate transporters which does not activate AMPA receptors or metabotropic glutamate receptors. Without wishing to be bound by any particular theory, this approach allows stimulation of a broad area of the cell, providing greater sensitivity than focal pressure application. To increase the probability of detecting transporter activity, experiments were performed with an internal solution containing SCN⁻, which is highly permeant through glutamate transporters.

Acute hippocampal slices (400μm) were prepared from P13-17 rats or mice in ice-cold solution containing choline chloride (110 mM), KCl (2.5 mM), CaCl2 (0.5 mM), MgSO4 (7 mM), NaH2PO4 (1.25 mM), choline bicarbonate (25 mM), D-Glucose (25 mM), Na− pyruvate (3.1 mM), and kynurenic acid (1 mM). Slices were allowed to recover at 37° for 30 min in artificial CSF containing NaCl (119 mM), KCl (2.5 mM), CaCl2 (2.5 mM), MgCl2 (1.3 mM), NaH2PO4 (1 mM), NaHCO3 (26.2 mM) and D-Glucose (11 mM), then maintained in a submerged chamber oxygenated with 95% O2/5% CO2. Whole cell recordings were made from pyramidal neurons using an internal solution containing KSCN (130 mM), EGTA (10 mM), HEPES (20 mM), MgCl2 (1 mM); pH 7.3. Currents were recorded with an Axopatch 200B amplifier, filtered at 3 kHz, then digitized using pClamp software. The UV output of a CW Argon Laser (Spectraphysics 2017) was focused to a 200 μm spot on the soma and apical dendrites of pyramidal neurons to initiate photolysis of MNI-D-aspartate (500 μM). Antagonists were used to block voltage-gated Na+ channels, AMPA/kainate receptors, NMDA receptors, and GABAA receptors. Glutamate transporters were inhibited using TBOA (200 mM). For each experiment, the caged compound solution contained the same concentration of antagonists present in the bath solution.

B. Results

Generation of GLAST- and GLT-1-BAC Promoter Reporter Mice

GLAST and GLT-1 BAC's were modified as shown in FIGS. 1-2, and transgenic mice were generated. Out of 62 potential founder pups for the GLAST-DsRed transgene, three were genotyped positive. Two of these lines, 593 and 570, demonstrated a much stronger reporter expression phenotype than the third, line 573, most likely because of a greater number of inserted head-to-tail copies of the transgene.

Similarly, out of 51 potential founder mice for the GLT-1-eGFP transgene, two were genotyped positive, with line 335 demonstrating a much stronger phenotype than line 356.

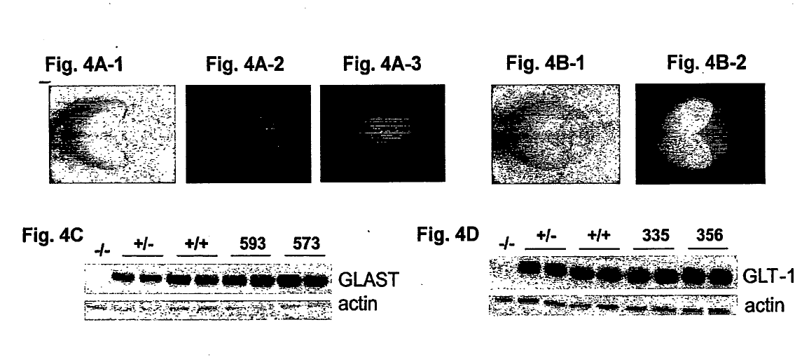

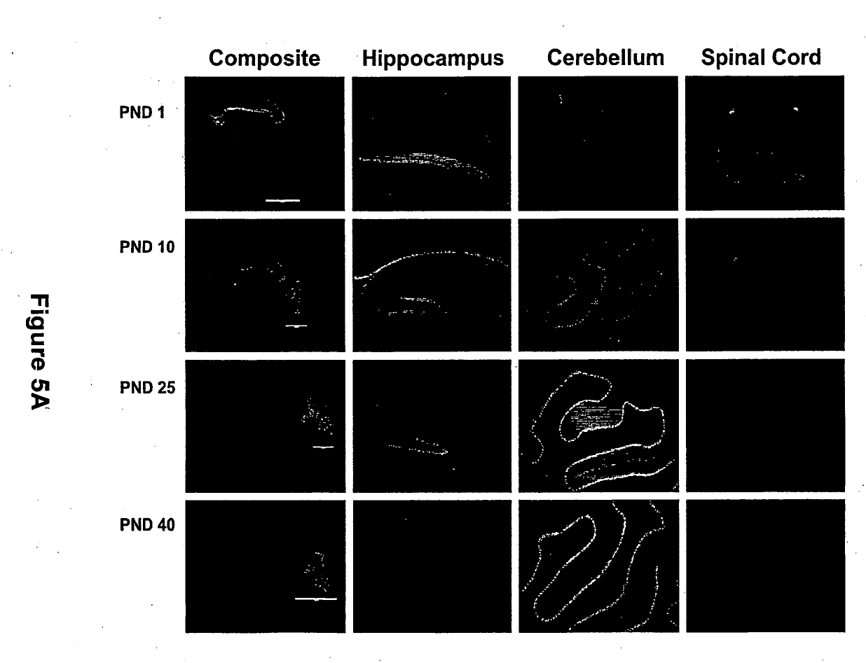

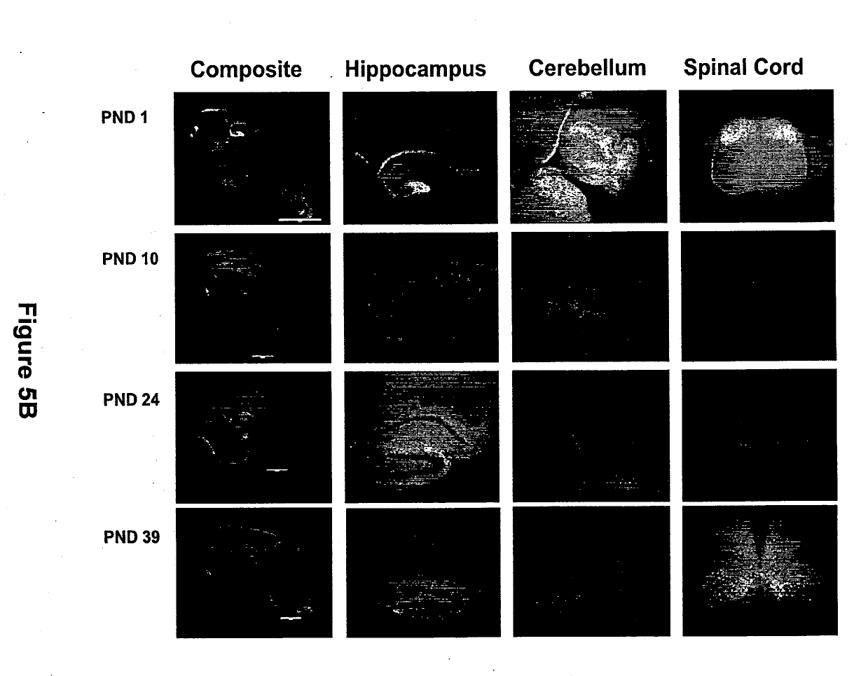

The gross promoter activation can be seen even at the whole brain level, as shown in FIG. 4. It was observed that the greatest activation of the GLAST promoter was in the cerebellum. In comparison, the greatest activation of the GLT-1 promoter was observed in the cortex, with a relatively lesser amount in the cerebellum.

Although the entire GLAST and GLT-1 genes are present in the BAC construct, GLAST or GLT-1 mRNA or protein was not produced by these transgenes because the first coding exon, in the case of GLAST, and the start codon, in the case of GLT-1, were interrupted by the reporter cDNA followed by a signal for a poly-A tail.

In addition, the respective transporter protein levels were observed to be unchanged in the BAC reporter transgenic mice as compared to wildtype mice, as shown in FIG. 4. Extra copies of the GLAST or GLT-1 promoters could serve as a sink for transcription elements, thus actually reducing the specific transporter protein levels in the transgenic mice. This would, in turn, make the mice less useful for studies of up- or down-regulation of the transporters. However, this was not the case. The Western blots in FIG. 4 demonstrate that there were equal amounts of GLAST/GLT-1 protein in the strong reporter lines (593, 335) and in their wild type littermates (+/+), as well as in the weak reporter lines (573, 356). As a control, total protein from knockout mammals (−/−) (Tanaka et al., 1997, Science 276:1699-1702; Watase et al., 1998, Eur. J. Neurosci. 10:976-988) heterozygote knockout mammals (+/−) was used. In each blot, the bands for actin indicated that loading from lane to lane was equal.

Developmental Changes in GLAST Promoter Activity

GLAST promoter activation in whole brain, hippocampus, cerebellum, and spinal cord at postnatal days 1, 10, 24, and 40 was inspected. As shown in the composite panels of FIG. 5A, it was observed that GLAST promoter activity in a newborn mouse, postnatal day (PND) 1 was highest in the cerebral cortex and periventricular progenitor proliferation zones. This activation in cortex was greatly reduced by PND 25, whereas the GLAST promoter was strongly active in radial glia of the hippocampal dentate gyrus and in Bergmann glia of the cerebellum in the adult (PND 40). Expression in the spinal cord was observed in white and gray matter, although more prominent in dorsal white matter columns throughout development. Histologically, the DsRed protein product tended to remain localized to the soma of cells, but could be seen occasionally in processes. Closer inspection by confocal microscopy indicated that the DsRed protein tended to form intra cellular aggregates.

Developmental Changes in GLT-1 Promoter Activity

Inspection of GLT-1-eGFP BAC reporter lines at postnatal days 1, 10, 24, and 40 revealed strong promoter activity throughout the CNS, primarily in astrocytes. Within the same image, or across images taken with the same microscope and camera settings, promoter activation levels can be directly compared because the level of eGFP expression is indicative of the relative levels of promoter activation. The localization of the eGFP reporter protein was observed to be throughout the soma and processes of cells (FIG. 5B).

### Example 2

### GLAST and GLT-1 Promoters are Activated in Different Brain Regions at Postnatal Days 1 and 24 (Double Transgenic)

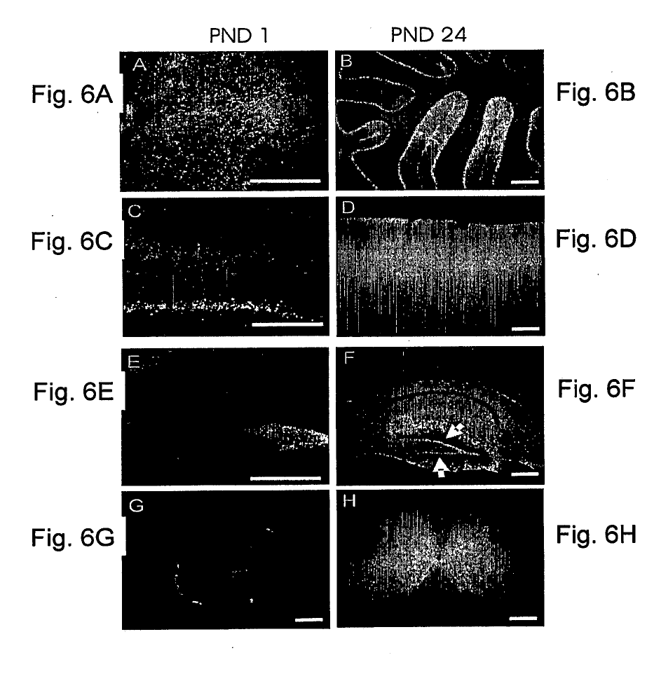

Past immunochemistry demonstrated that astrocytes may express both GLAST and GLT-1. To investigate the cellular expression patterns of GLT-1 and GLAST, GLAST-DsRed and GLT-1-eGFP BAC transgenic mice were crossed to create double transgenic mice. These double transgenic mice were used for identification of cells capable of both GLAST and GLT-1 expression. These double transgenic mice offered the opportunity to view GLT-1 and GLAST promoter activation levels in parallel. Grossly, at both low power and even higher power microscopy, there were few examples of overlap between GLAST and GLT-1 expression. Images from specific brains regions at PND's 1 and 24 are shown in FIG. 6. In cerebellum (FIG. 6A and FIG. 6B), GLAST promoter activation was observed in the external granule layer at PND 1 and primarily in the Bergmann glia by PND 24, whereas the GLT-1 promoter was consistently activated. In cerebral cortex, both promoters were strongly active at PND 1 (FIG. 6C) but in different layers of the cortex. By PND 24 (FIG. 6D), GLT-1 expression was dominant in the cortex, with the exception of layer 6, the region adjacent to ventricles where some GLAST promoter activity was visible. Expression of GLAST in this periventricular region was abolished in adult animals.

In hippocampus, at PND 1 (FIG. 6E), both promoters were active and prominent. At PND 24 (FIG. 6F), GLT-1 promoter activity was dominant in the hippocampus except for in the subgranular layer of the dentate gyrus (white arrows) where both promoters were active.

In spinal cord (FIG. 6G and FIG. 6H), there was little overlap between promoter activation of GLAST and GLT-1, with GLT1 expression widely seen in gray matter. GLAST promoter reporter was seen in gray matter but was more strongly present in white matter tracts. Importantly, dual expressing cells were rarely seen in brain or spinal cord. Close inspection by confocal microscopy revealed complete independent expression in both white and gray matter of the spinal cord. Similar independent expression of GLAST and GLT-1 was seen in cortex, striatum, and brainstem. However, distinct dual expression was observed in the dentrate gyrus of the hippocampus and in cerebellar radial glia.

### Example 3

### GLAST and GLT-1 Promoters are Active in Distinct Subsets of Cells

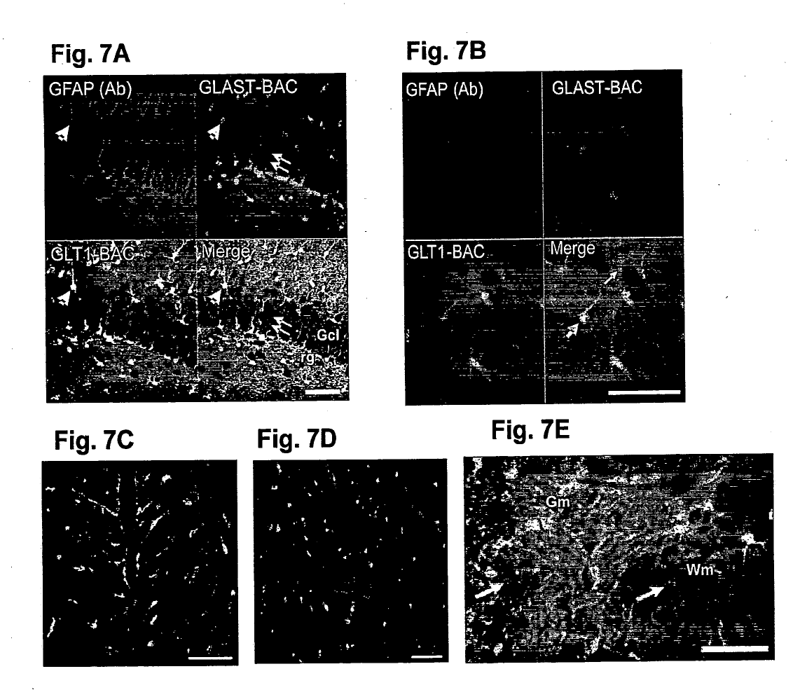

In order to explore the cell types demonstrating both overlapping and non-overlaping overlaping activation of GLAST and GLT-1 promoters, GFAP marker to identify astroctyes were added to the confocal images of several regions at PND 24. Some of these studies were performed on tissues obtained from double transgenic animals expressing both the GLAST-DSRed promoter reporter and the GLT1-eGFP promoter reporter. As shown in FIG. 7A, both GLAST and GLT-1 promoters were active in GFAP positive astrocytes of the hippocampus dentate gyrus (yellow arrows), as well as in the radial glia, but there were also cells in the granule cell layer (gcl) in which the GLAST promoter was active independent of the GLT-1 promoter or GFAP protein (white arrows). In a higher magnification view of cells in the hippocampus CA3 region, shown in FIG. 7B, it was apparent that GFAP positive astrocytes demonstrated GLAST and GLT-1 promoter activation (yellow arrow). However, there was also obvious non-astrocytic GLAST expressing cells (white arrow).

In PND 24 spinal cord from double transgenic animals, there was little overlap between GLT-1 and GLAST promoter activation in the ventral white column (FIG. 7C), dorsal white column (FIG. 7D), nor at the white-gray junction, as shown in FIG. 7E, indicating that these promoters were active in distinct subsets of cells. However, the GLAST positive cells were not GFAP positive (FIG. 7E).

### Example 4

### The GLAST Promoter is Active in Oligodendroglia and a Subset of Unidentified Cells

By PND 24, all GFAP positive astrocytes appeared to also be positive for GLT-1 promoter activation, and vice versa, in all regions examined. However, as shown in FIG. 7, this was not always the case with GLAST promoter activation. While the GLAST promoter was activated in some of the GFAP/GLT-1 positive cells (especially in cerbellar cortex and hippocampus), it was also active in non-astrocytic cells. Therefore markers for other cell types were stained in several brain regions in order to identify what these other cell types were.

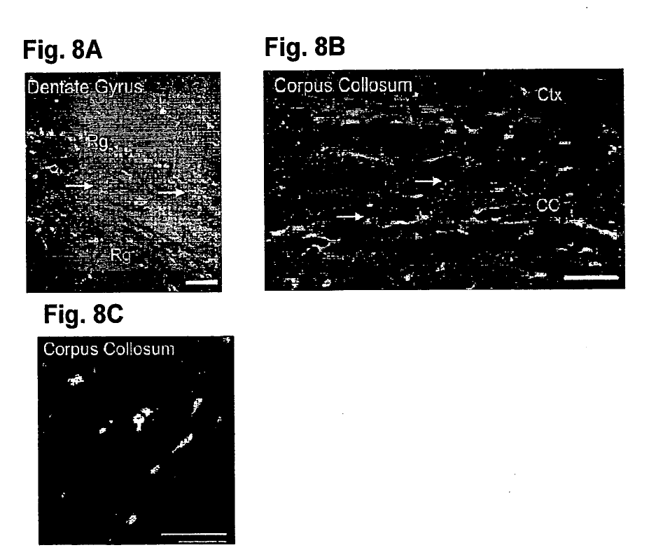

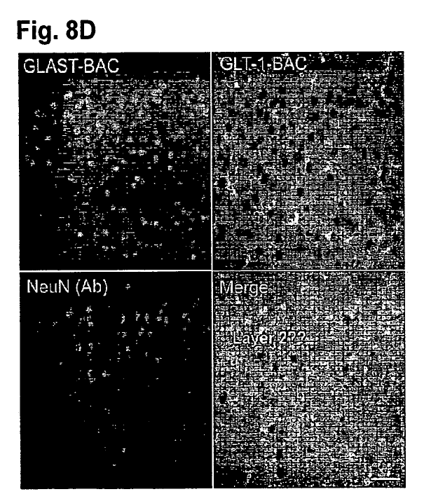

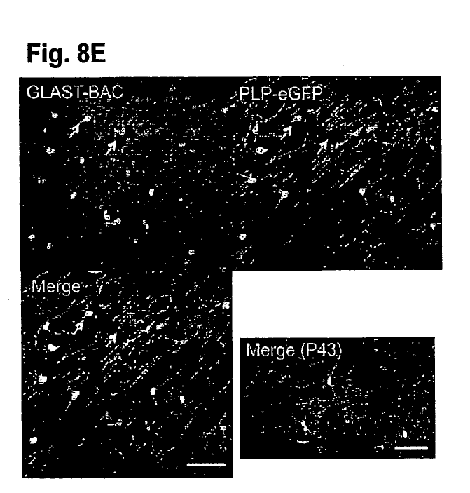

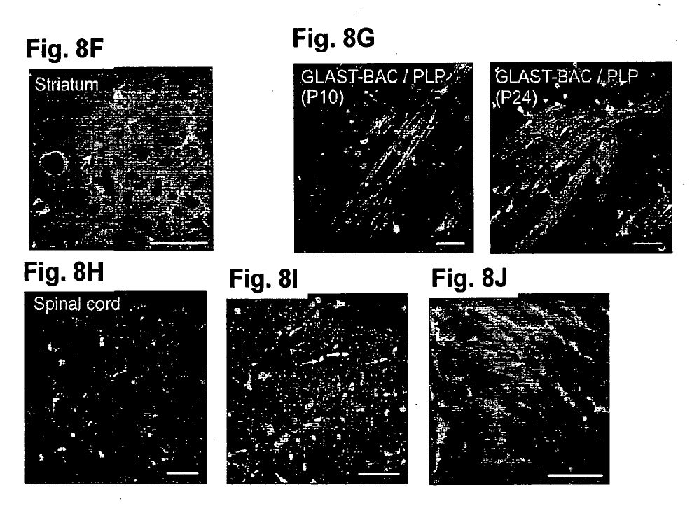

FIG. 8A shows that GLT-1 and GLAST promoters were both active in the radial glia of the dentate gyus. Another small population of GLAST promoter-active positive cells, negative for GLT-1 promoter activation and for the neuronal marker NeuN (blue), was observed in the granule cell layer.

In the corpus callosum (FIG. 8B), the vast majority of cells were negative for both GLT-1 promoter expression and neuronal markers. The next set of experiments were to address whether these cells might be oligodendrocytes by using the oligodendrocyte marker myelin proteolipid protein (PLP). To evaluate the overlap between GLAST expression and oligodenroglia, GLAST-DsRed BAC promoter reporter mice were mated to PLP-eGFP promoter reporter mice (Fuss et al., Dev. Biol. 218:259-274). An image of the corpus callosum at PND 24, shown in FIG. 8C demonstrated that few, if any, of GLAST promoter reporter cells are oligodendroglia. Thus, some of the GLAST promoter active cells in the corpus callosum were neither PLP-positive, nor were they GLT-1 promoter positive.

There were also non-GLT-1 promoter activated GLAST cells in cortex. These were small round cells that appeared to be neurons or neuron progenitors based on co-staining with the NeuN antibody, as shown in FIG. 8D. They were typically periventricular and likely late remnants of the periventricular progenitor cells. The number of these GLAST promoter-active/NeuN positive cells were high at PND 10 and gradually decreased with age until there were only a few that could be seen at PND 49 and none visible at PND 58.

A large number of GLAST-promoter active cells in the cortex also overlapped with oligodendrocytes (PLP promoter reporter positive) at PND 24 and PND 43, as shown in FIG. 8E, but not at P10.

The localization of GLAST promoter activation to oligodendroglia was unexpected. Since a number of the GLAST positive cells were not astrocytes or neurons, the next set of experiments were set out to determine if they could overlap with the widely prevelant NG2 cells, oligodrondrocyte precursors. However, co-staining with the NG2 antibody did not reveal overlap with any of the GLAST promoter reporter cells in any brain region. This fits with other physiological data that is unable to detect glutamate transporter currents in NG2 cells.

In the striatum, the majority of GLAST promoter-active cells were not astrocytes, as determined by confocal GFAP immunoreactivity and GLT1-promoter reporter analysis (FIG. 8F). However, many GLAST promoter-active cells overlapped with the PLP oligodendrocyte promoter reporter at PND 24 (FIG. 8G) and PND 43, although this expression was not seen in early postnatal animals (FIG. 8G).

In the spinal cord (FIG. 8), the expression of GLT-1 and GLAST promoter reporters was completely separate, with the vast majority of gray matter astrocytes demonstrating GLT-1 promoter activation. The GLAST expressing cells were pronounced in the white matter tracts, and many of these cells appeared to be oligodendroglia (8I, white arrows), but not neurons (8H) or microglia (8J).

In multiple brain regions, GLAST positive/GLT-1 negative cells were often seen together, adjacent to neuronal somas. These cells were not oligodroglia (were PLP negative) and did not stain for GFAP or microglial markers. These GLAST positive/GLT-1 negative cells are believed to be perineuronal glia.

The results presented herein demonstrate that GLAST, unlike GLT-1, appears to be expressed by progenitor cells in the early postnatal period. It was observed that GLAST positive cells were intensely localized to the periventricular white and gray matter in early postnatal time points. Even at later postnatal time points, GLAST was expressed by NeuN positive cells periventricularly, althougth they had fully disappeared in adult brain.

The invention has been described in detail with reference to preferred embodiments thereof. However, it will be appreciated that those skilled in the art, upon consideration of this disclosure, may make modification and improvements within the spirit and scope of the invention as set forth in the following claims.

1. A non-human transgenic animal comprising a bacterial artificial chromosome transgene, wherein:
a) the bacterial artificial chromosome comprises genomic DNA comprising a locus selected from the group consisting of the GLT-1 locus and the GLAST locus;
b) the locus comprises a reporter gene that is operatively linked to the promoter of the locus.

2. The non-human transgenic animal of claim 1, wherein said animal is a mammal.

3. The non-human transgenic animal of claim 2, wherein said mammal is a rodent.

4. The non-human transgenic animal of claim 1, wherein the bacterial artificial chromosome comprises bacterial artificial chromosome clone RPCI-23-361H22.

5. The non-human transgenic animal of claim 1, wherein the bacterial artificial chromosome comprises bacterial artificial chromosome clone RPCI-24-287G11.

6. The non-human transgenic animal of claim 1, wherein the reporter gene comprises a nucleic acid sequence encoding at least one protein selected from the group consisting of luciferase, β-galactosidase, chloramphenicol acetyl transferase and a fluorescent protein.

7. The non-human transgenic animal of claim 6, wherein the luciferase is selected from the group consisting of firefly luciferase and Renilla luciferase.

8. The non-human transgenic animal of claim 6, wherein the fluorescent protein is selected from the group consisting of green fluorescent protein, enhanced green fluorescent protein, red fluorescent protein, yellow fluorescent protein, blue fluorescent protein and cyan fluorescent protein.

9. A cell isolated from a non-human transgenic animal comprising a bacterial artificial chromosome transgene, wherein:
a) the bacterial artificial chromosome comprises genomic DNA comprising a locus selected from the group consisting of the GLT-1 locus and the GLAST locus;
b) the locus comprises a reporter gene that is operatively linked to the promoter of the locus.

10. The cell of claim 9, wherein said cell is a primary cell.

11. The cell of claim 9, wherein said cell is immortalized.

12. The cell of claim 9, wherein said cell is an astrocyte.

13. The cell of claim 12, wherein said cell exhibits bacterial artificial chromosome GLT-1 promoter activity.

14. The cell of claim 9, wherein said cell is an oligodendrocyte.

15. The cell of claim 14, wherein said cell exhibits bacterial artificial chromosome GLAST promoter activity.

16. The cell of claim 9, wherein said cell exhibits bacterial artificial chromosome GLT-1 and bacterial artificial chromosome GLAST promoter activity.

17. A method of identifying a compound capable of treating a neurological disorder comprising:
a) contacting a test compound with a cell isolated from a non-human transgenic animal comprising a bacterial artificial chromosome transgene, wherein the bacterial artificial chromosome comprises genomic DNA comprising a locus selected from the group consisting of the GLT-1 locus and the GLAST locus, and the locus comprises a reporter gene that is operatively linked to the promoter of the locus; and
b) determining whether expression of the reporter gene is modulated, thereby identifying a compound which modulates expression of the reporter gene as a compound which is capable of treating a neurological disorder.

18. The method of claim 17, wherein expression of the reporter gene is upregulated.

19. The method of claim 17, wherein expression of the reporter gene is downregulated.

20. A method of identifying a compound capable of treating a psychiatric disorder comprising:
a) contacting a test compound with a cell isolated from a non-human transgenic animal comprising a bacterial artificial chromosome transgene, wherein the bacterial artificial chromosome comprises genomic DNA comprising a locus selected from the group consisting of the GLT-1 locus and the GLAST locus, and the locus comprises a reporter gene that is operatively linked to the promoter of the locus; and
b) determining whether expression of the reporter gene is modulated, thereby identifying a compound which modulates expression of the reporter gene as a compound which is capable of treating a psychiatric disorder.

21. The method of claim 20, wherein expression of the reporter gene is upregulated.

22. The method of claim 20, wherein expression of the reporter gene is downregulated.

23. A method of identifying a compound capable of treating a neurological disorder comprising:
a) contacting a test compound with a non-human transgenic animal comprising a bacterial artificial chromosome transgene, wherein the bacterial artificial chromosome comprises genomic DNA comprising a locus selected from the group consisting of the GLT-1 locus and the GLAST locus, and the locus comprises a reporter gene that is operatively linked to the promoter of the locus; and
b) determining whether expression of the reporter gene is modulated, thereby identifying a compound which modulates expression of the reporter gene as a compound which is capable of treating a neurological disorder.

24. The method of claim 23, wherein expression of the reporter gene is upregulated.

25. The method of claim 23, wherein expression of the reporter gene is downregulated.

26. A method of identifying a compound capable of treating a psychiatric disorder comprising:
a) contacting a test compound with a non-human transgenic animal comprising a bacterial artificial chromosome transgene, wherein the bacterial artificial chromosome comprises genomic DNA comprising a locus selected from the group consisting of the GLT-1 locus and the GLAST locus, and the locus comprises a reporter gene that is operatively linked to the promoter of the locus; and
b) determining whether expression of the reporter gene is modulated, thereby identifying a compound which modulates expression of the reporter gene as a compound which is capable of treating a a psychiatric disorder.

27. The method of claim 26, wherein expression of the reporter gene is upregulated.

28. The method of claim 26, wherein expression of the reporter gene is downregulated.

29. A method of treating a mammal suffering from a neurological disease or disorder, the method comprising administering to said mammal a compound capable of increasing the activity of a glutamate transporter promoter, wherein said glutamate transporter promoter is selected from the group consisting of GLT-1 and GLAST, and wherein said compound is identified by the method of claim 17 or claim 23.

30. A method of treating a mammal suffering from a psychiatric disease or disorder, the method comprising administering to said mammal a compound capable of increasing the activity of a glutamate transporter promoter, wherein said glutamate transporter promoter is selected from the group consisting of GLT-1 and GLAST, and wherein said compound is identified by the method of claim 20 or claim 26.

31. A method of isolating a cell from a non-human transgenic animal comprising a bacterial artificial chromosome transgene, wherein the bacterial artificial chromosome comprises genomic DNA comprising a locus selected from the group consisting of the GLT-1 locus and the GLAST locus;
wherein said locus comprises a reporter gene that is operatively linked to the promoter of the locus, the method comprising:
providing an antibody specific for said reporter gene;
contacting said population of cells with said antibody under conditions suitable for formation of an antibody-cell complex;
and substantially separating said antibody-cell complex from said population of cells; thereby isolating said cell.

32. The method of claim 31, wherein said antibody is conjugated to a physical support.

33. The method of claim 31, wherein said physical support is selected from the group consisting of a microbead, a magnetic bead, a panning surface, a dense particle for density centrifugation, an adsorption column, and an adsorption membrane.

34. The method of claim 31, wherein said physical support is selected from the group consisting of a streptavidin bead and a biotin bead.

35. The method of claim 31, wherein said antibody-cell complex is substantially separated from said population of cells using a method selected from the group consisting of fluorescence activated cell sorting (FACS) and magnetic activated cell sorting (MACS).

36. A non-human double transgenic animal comprising a first and a second bacterial artificial chromosome transgene, wherein the first bacterial artificial chromosome comprises a GLT-1 genomic DNA operatively linked to a first reporter gene, and wherein the second bacterial artificial chromosome comprises a GLAST genomic DNA operatively linked to a second reporter gene.

37. A cell isolated from a non-human double transgenic animal comprising a first and a second bacterial artificial chromosome transgene, wherein the first bacterial artificial chromosome comprises a GLT-1 genomic DNA operatively linked to a first reporter gene, and wherein the second bacterial artificial chromosome comprises a GLAST genomic DNA operatively linked to second reporter gene.
